(19) United States
(12) Patent Application Publication
(10) Pub. No.: US 2026/0325520 A1
(43) Pub. Date: Oct. 22, 2026

(54) METHODS FOR TREATING OR PREVENTING NEUROENDOCRINE TUMR FORMATION USING CDC7 INHIBITORS

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Álvaro D. QUINTANAL VILLALONGA, New York, NY (US); Charles Michael RUDIN, New York, NY (US)

(21) Appl. No.: 19/121,406

(22) PCT Filed: Oct. 16, 2023
(86) PCT No.: PCT/US2023/076996

§ 371 Date: Apr. 15, 2025

Related U.S. Application Data
(60) Provisional application No. 63/416,702, filed on Oct. 17, 2022.

(51) Int. Cl. A61K 31/519; A61K 31/05; A61K 31/167; A61K 31/277; A61K 31/403; A61K 31/4155; A61K 31/4166; A61K 31/4439; A61K 31/444; A61K 31/4709; A61K 31/506; A61K 31/517; A61K 31/5377; A61K 31/58; A61K 33/243; A61K 45/06; A61P 35/00; C07K 16/28

(52) U.S. Cl. CPC A61K 31/519; A61K 31/05; A61K 31/167; A61K 31/277; A61K 31/403; A61K 31/4155; A61K 31/4166; A61K 31/4439; A61K 31/444; A61K 31/4709; A61K 31/506; A61K 31/517; A61K 31/5377; A61K 31/58; A61K 33/243; A61K 45/06; A61P 35/00; C07K 16/2863; C07K 2317/76

(57) ABSTRACT

The present disclosure provides methods for treating or preventing neuroendocrine tumor formation in subjects diagnosed with TP53 and RB1 deficient adenocarcinomas (e.g., lung or prostate adenocarcinomas) using CDC7 inhibitors. Also disclosed herein are methods for preventing neuroendocrine tumor formation in subjects diagnosed with adenocarcinomas (e.g., TP53 and RBI deficient lung or prostate adenocarcinomas) using CDC7 inhibitors in combination with androgen receptor (AR) inhibitors, epidermal growth factor receptor (EGFR) inhibitors, or chemotherapeutic drugs.

### CROSS-REFERENCE TO RELATED APPLICATIONS

This application claims the benefit of and priority to U.S. Provisional Patent Application No. 63/416,702 filed Oct. 17, 2022, the entire contents of which are incorporated herein by reference.

### STATEMENT OF GOVERNMENT SUPPORT

This invention was made with government support under CA197936, and U24 CA213274, CA264078-01 and CA08748, awarded by the National Institutes of Health. The government has certain rights in the invention.

### TECHNICAL FIELD

The present disclosure provides methods for treating or preventing neuroendocrine tumor formation in subjects diagnosed with TP53 and RB1 deficient adenocarcinomas (e.g., lung or prostate adenocarcinomas) using CDC7 inhibitors. Also disclosed herein are methods for preventing neuroendocrine tumor formation in subjects diagnosed with adenocarcinomas (e.g., TP53 and RB1 deficient lung or prostate adenocarcinomas) using CDC7 inhibitors in combination with androgen receptor (AR) inhibitors, epidermal growth factor receptor (EGFR) inhibitors, or chemotherapeutic drugs.

### BACKGROUND

The following description of the background of the present technology is provided simply as an aid in understanding the present technology and is not admitted to describe or constitute prior art to the present technology.

Lineage plasticity, the capacity of cells to transition from one committed identity to that of a distinct developmental lineage, can promote survival of cancer cells under unfavorable conditions such as oncogenic driver-targeted therapy. Under the selective pressure of targeted therapies, histological transformation of adenocarcinoma (AD) to highly aggressive neuroendocrine (NE) derivatives resembling small cell carcinomas has been reported in up to 20% of AR-dependent prostate cancers and up to 14% of EGFR-mutant lung ADs. It has been described that tumors with both TP53 and RB1 mutations/loss show increased susceptibility to transformation, thus defining a patient population at risk. NE transformation in both disease contexts is associated with notably poor prognoses. Little is known about the molecular alterations driving NE transformation in human tumors, in part due to the absence of viable models to study this phenomenon, and in part to the paucity of transformation samples available for molecular analysis. To date (1) no specific therapies for NE transformation prevention are available for patients at high risk of transformation, and (2) the primary therapy available for NE-transformed patients, platinum doublet (cisplatin and etoposide, plus immunotherapy in the lung setting), show only short-term responses.

Accordingly, there is an urgent need for effective therapies for treating or preventing NE transformation in adenocarcinoma patients.

### SUMMARY OF THE PRESENT TECHNOLOGY

In one aspect, the present disclosure provides a method for treating or preventing neuroendocrine tumor formation in a patient diagnosed with adenocarcinoma comprising administering to the patient an effective amount of a CDC7 inhibitor, wherein the adenocarcinoma exhibits reduced expression and/or activity of TP53 and RB1. In some embodiments, the adenocarcinoma comprises (a) a genetic mutation, an indel, a copy number alteration or epigenetic downregulation in TP53 and (b) a genetic mutation, an indel, a copy number alteration or epigenetic downregulation in RB1.

In another aspect, the present disclosure provides a method for preventing neuroendocrine tumor formation in a patient diagnosed with adenocarcinoma comprising administering to the patient an effective amount of a CDC7 inhibitor and an effective amount of at least one chemotherapeutic drug, wherein the adenocarcinoma exhibits reduced expression and/or activity of TP53 and RB1. Also provided herein is a method for enhancing responsiveness of a patient with neuroendocrine tumors to systemic chemotherapy comprising administering to the patient an effective amount of a CDC7 inhibitor and an effective amount of at least one chemotherapeutic drug, wherein the neuroendocrine tumors exhibit reduced expression and/or activity of TP53 and RB1. In some embodiments, the adenocarcinoma or the neuroendocrine tumors comprise(s) (a) a genetic mutation, an indel, a copy number alteration or epigenetic downregulation in TP53 and (b) a genetic mutation, an indel, a copy number alteration or epigenetic downregulation in RB1. In some embodiments, the CDC7 inhibitor and the at least one chemotherapeutic drug are administered sequentially, simultaneously, or separately. Additionally or alternatively, in some embodiments, the at least one chemotherapeutic drug is administered orally, intranasally, parenterally, intravenously, intramuscularly, intraperitoneally, or subcutaneously, intratumorally, or topically. The at least one chemotherapeutic drug may be an alkylating agent, a platinum agent, a taxane, a vinca agent, an aromatase inhibitor, a cytostatic alkaloid, a cytotoxic antibiotic, an antimetabolite, an endocrine/hormonal agent, or a bisphosphonate therapy agent. Examples of chemotherapeutic drugs include, but are not limited to cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, risedronate, pamidronate, ibandronate, alendronate, denosumab, zoledronate, tykerb, anthracyclines (e.g., daunorubicin and doxorubicin), oxaliplatin, melphalan, etoposide, mechlorethamine, bleomycin, microtubule poisons, annonaceous acetogenins, abraxane, leucovorin, nab-paclitaxel, everolimus, pegylated-hyaluronidase, pemetrexed, folinic acid, MK2206, GDC-0449, IPI-926, M402, LY293111 or combinations thereof.

In yet another aspect, the present disclosure provides a method for preventing neuroendocrine tumor formation in a patient diagnosed with adenocarcinoma comprising administering to the patient an effective amount of a CDC7 inhibitor and an effective amount of an androgen receptor (AR) inhibitor. The adenocarcinoma may exhibit reduced expression and/or activity of TP53 and RB1. In certain embodiments, the adenocarcinoma comprises (a) a genetic mutation, an indel, a copy number alteration or epigenetic downregulation in TP53 and (b) a genetic mutation, an indel, a copy number alteration or epigenetic downregulation in RB1. Additionally or alternatively, in some embodiments, the CDC7 inhibitor and the AR inhibitor are administered sequentially, simultaneously, or separately. Additionally or alternatively, in some embodiments, the AR inhibitor is administered orally, intranasally, parenterally, intravenously, intramuscularly, intraperitoneally, or subcutaneously, intratumorally, or topically. Examples of AR inhibitors include, but are not limited to apalutamide, bicalutamide, darolutamide, enzalutamide, flutamide, abiraterone acetate, ARN-509, and nilutamide.

In another aspect, the present disclosure provides a method for preventing neuroendocrine tumor formation in a patient diagnosed with adenocarcinoma comprising administering to the patient an effective amount of a CDC7 inhibitor and an effective amount of an epidermal growth factor receptor (EGFR) inhibitor (EGFRi) (e.g., EGFR tyrosine kinase inhibitor (TKI), anti-EGFR antibodies). The adenocarcinoma may exhibit reduced expression and/or activity of TP53 and RB1. In certain embodiments, the adenocarcinoma comprises (a) a genetic mutation, an indel, a copy number alteration or epigenetic downregulation in TP53 and (b) a genetic mutation, an indel, a copy number alteration or epigenetic downregulation in RB1. Additionally or alternatively, in some embodiments, the CDC7 inhibitor and the EGFRi are administered sequentially, simultaneously, or separately. Additionally or alternatively, in some embodiments, the EGFRi is administered orally, intranasally, parenterally, intravenously, intramuscularly, intraperitoneally, or subcutaneously, intratumorally, or topically. Examples of EGFRis include, but are not limited to, osimertinib, afatinib, erlotinib, gefitinib, icotinib, dacomitinib, rociletinib, olmutinib, cetuximab, panitumumab, nimotuzumab, and necitumumab.

In any and all embodiments of the methods disclosed herein, the patient is diagnosed with TP53⁻/⁻and RB1⁻/⁻mutant adenocarcinoma. In some embodiments, the TP53⁻/⁻and RB1⁻/⁻mutant adenocarcinoma is lung adenocarcinoma or prostate adenocarcinoma. Additionally or alternatively, in some embodiments, the CDC7 inhibitor is administered orally, intranasally, parenterally, intravenously, intramuscularly, intraperitoneally, or subcutaneously, intratumorally, or topically. Examples of CDC7 inhibitors include, but are not limited to simurosertib (TAK-931), PHA-767491, carvedilol, dequalinium chloride, ticagrelor, and clofoctol.

In any and all embodiments of the methods disclosed herein, the patient is human. Additionally or alternatively, in some embodiments, the patient is non-responsive to at least one prior line of cancer therapy such as chemotherapy.

### BRIEF DESCRIPTION OF THE DRAWINGS

FIGS. 1A-1J demonstrate that CDC7 is highly expressed in SCLC and exert pro-oncogenic effects in this setting. FIG. 1A: CDC7 mRNA expression in cell lines derived from different tumor types. The data was obtained from CCLE through UCSC Xenabrowser portal (https://xenabrowser.net/) in December 2020. Lines indicate the median CDC7 mRNA expression in SCLC cell lines. FIG. 1B: CDC7 protein expression assessed by IHC in NSCLC (N=58) and SCLC (N=45) clinical samples. Protein expression is shown as IHC score. H-score medians and standard deviation are shown. FIG. 1C: Western blot showing CDC7 KO in H82 (SCLC-N) and H146 (SCLC-A) SCLC cell lines. Proliferation (FIG. 1D) and soft agar colony formation (FIG. 1E) assays in isogenic H82 and H146 cell lines with CDC7 KO. In FIG. 1D, lower line graphs correspond to CDC7 KO (sgCDC7). FIG. 1F: Ectopic overexpression of CDC7 in H69 (SCLC-A) and DMS114 (SCLC-Y) SCLC cell lines. Proliferation (FIG. 1G) and soft agar colony formation (FIG. 1H) assays in isogenic H69 and DMS114 cell lines with ectopic CDC7 overexpression. Student's t-test was performed to assess statistical significance (two-tailed, assuming heterogeneous value distribution). In FIG. 1G, top line graphs correspond to CDC7 overexpression. p-value legend: *<0.05, **<0.01, ***<0.001. FIG. 1I: Bivariate correlation of CDC7 mRNA and protein expression with simurosertib or LY3143921 sensitivity (assessed as growth inhibition) in an array of SCLC cell lines. Correlation was calculated with Spearman's test. FIG. 1J: Plot showing viability of control and CDC7 CRISPR-Cas9 KO cell lines using two different sgRNAs (sg1 and sg2) after treatment with simurosertib GI50 for 5 days. Viability is normalized to the untreated condition for each experimental condition (control, sg1 and sg2). Student's t-test was performed to assess statistical significance (two-tailed, assuming heterogeneous value distribution) p-value legend: *<0.05, **<0.01, **<0.001.

FIGS. 2A-2H demonstrate that CDC7 inhibition strongly sensitizes SCLC to chemotherapy. FIG. 2A: CDC7 protein expression, shown as H-score, assessed by IHC in treatment-naive (N=39) and pre-treated (N=6) clinical samples. H-score medians and standard deviation are shown. FIG. 2B: Proliferation assays of untreated and cisplatin-treated (GI20 concentration) H82 and H146 cell lines with endogenous CDC7 expression versus CDC7 KO. FIG. 2C: Synergy plots showing the occurrence of synergy (red), addition (white) or antagonism (green) of the different combinations of simurosertib and cisplatin or irinotecan, calculated with the HSA method using the SynergyFinder web application (2.0). FIG. 2D: CDC7 protein expression, shown as H-score, assessed by IHC in an array of SCLC PDXs derived from chemotherapy-naïve and -treated tumors. FIG. 2E: Tumor growth curves of chemotherapy-naïve SCLC PDXs with high (Lx1231), intermediate (Lx33) and low (Lx276) CDC7 protein expression treated with cisplatin, etoposide, simurosertib or their combinations. FIG. 2F: Tumor growth curves of SCLC PDXs derived from pre-treated tumors with high (Lx761c), intermediate (Lx674c) and low (Lx95) CDC7 protein expression treated with irinotecan, simurosertib or their combinations. Student's t-test was performed to assess statistical significance (two-tailed, assuming heterogeneous value distribution). p-value legend: *<0.05, **<0.01, ***<0.001. FIG. 2G: Representative experiment for apoptosis (Annexin V/PI) assay of H82 and H146 SCLC cell lines treated with cisplatin (cis), simurosertib (simu) or their combination (combo). FIG. 2H: Mouse weight measurements of in vivo treatments shown in FIG. 3G.

FIGS. 3A-3J demonstrate that CDC7 is upregulated during NE transformation in prostate and lung tumors and its inhibition sensitizes NE-transformed tumors to chemotherapy. CDC7 mRNA (FIG. 3A) and protein (FIG. 3B) expression in lung tumor clinical specimens, categorized as control never transformed adenocarcinomas (LUAD), transforming adenocarcinomas (T-LUAD) and small cell carcinomas (T-SCLC) and control de novo small cell carcinomas (SCLC). For FIG. 3B, H-score medians and standard deviation are shown. FIG. 3C: CDC7 mRNA expression in PRAD tumors with or without NE features. Data from Abida et al., PNAS 2019. FIG. 3D: CDC7 protein expression in PRAD and NEPC clinical specimens, as assessed by IHC. H-score medians and standard deviation and representative images (FIG. 3E) are shown. FIG. 3F: In vitro synergy assays in Lx1042 (T-SCLC) and H660 (NEPC) cell lines of the combination of simurosertib and cisplatin with average synergy score displayed, as assessed by ZIP and calculated using the SynergyFinder web application (2.0). FIG. 3G: In vivo treatment of Lx1042 (T-SCLC) and LuCAP49 (NEPC) PDXs was conducted to compare the efficacy of the combination of cisplatin and simurosertib versus that of cisplatin and etoposide. Student's t-test was performed to assess statistical significance (two-tailed, assuming heterogeneous value distribution) p-value legend: *<0.05, **<0.01, ***<0.001. FIG. 3H: CDC7 mRNA expression in adenocarcinoma clinical specimens, categorized by their TP53/RB1 status. Data obtained from LUAD TCGA (PanCancer), LUAD OncoSG (OncoSG, Nat Genetics 2020) and PRAD TCGA (PanCancer). FIG. 3I: DNA accessibility ATACseq data from isogenic control and TP53/RB1-loss of function H1563 and 22PC isogenic cell lines. The transcription start site for the CDC7 gene is highlighted. FIG. 3J: Plot showing a representative biological replicate of an experiment assessing viability of control and TP 53/RB1-loss H1563 and 22PC cells treated with 0.5 μM simurosertib for 5 days. Student's t-test was performed to assess statistical significance (two-tailed, assuming heterogeneous value distribution). p-value legend: *<0.05, **<0.01, ***<0.001.

FIGS. 4A-4C demonstrate that CDC7 upregulation after loss of TP53/RB1 function induces sensitivity to simurosertib. FIG. 4A: CDC7 mRNA expression in adenocarcinoma clinical specimens, categorized by their TP53/RB1 status. Data obtained from LUAD TCGA (PanCancer), LUAD OncoSG (OncoSG, Nat Genetics 2020) and PRAD TCGA (PanCancer). FIG. 4B: Western blot showing CDC7 protein levels in isogenic H1563 (LUAD) and 22PC (PRAD) cell lines with or without induced loss of function of TP53 and/or RB1 (see methods). FIG. 4C: Plot showing a representative biological replicate of an experiment assessing viability of control and TP53/RB1-loss H1563, 22PC and LnCap (PRAD) cells treated with 0.5 μM simurosertib. Student's t-test was performed to assess statistical significance (two-tailed, assuming heterogeneous value distribution). p-value legend: *<0.05, **<0.01, ***<0.001.

FIGS. 5A-5E demonstrate that CDC7 inhibition attenuated NE transformation in the prostate and lung. FIG. 5A: In vivo treatment of cell line xenografts for TP53/RB1-inactivated LnCap and 22PC cells with enzalutamide, simurosertib or their combination. FIG. 5B: Bar plot showing percentage of tumor classified as PRAD or NEPC, divided by treatment category, from (FIG. 5A) collected and endpoint for each of the groups. Average percentages and SEM is shown, per histology, per treatment group. Plots showing average and SEM protein expression, quantified as H-score, of NE markers (FIG. 5C) or AR (FIG. 5D) in tumors from (FIG. 5A) collected at endpoint for each of the groups. Analyses in FIGS. 5B-5D could not be performed in the 22PC model because combo-treated tumors were too small to collect. FIG. 5E: In vivo treatment of the EGFR-mutant combined NSCLC/SCLC MSK_Lx151 PDX with osimertinib, simurosertib or their combination. Student's t-test was performed to assess statistical significance (two-tailed, assuming heterogeneous value distribution) p-value legend: *<0.05, **<0.01, ***<0.001. FIG. 5E: In vivo treatment of the MSK_Lx151 PDX with osimertinib, simurosertib or their combination. Student's t-test was performed to assess statistical significance (two-tailed, assuming heterogeneous value distribution) p-value legend: *<0.05, **<0.01, ***<0.001.

FIGS. 6A-6E demonstrate that CDC7 inhibition attenuated NE transformation in the prostate and lung by activating the proteasome and inducing MYC degradation. FIG. 6A: Pathway enrichment analyses on differentially expressed genes between the combo-and enzalutamide-treated tumors, collected at an intermediate time point in the for TP53/RB1-inactivated LnCap and 22PC experiments shown in FIG. 5. FIG. 6B: Western blot showing MYC upregulation induced by targeted therapy (either enzalutamide or osimertinib), and MYC downregulation by simurosertib or the combination of targeted therapy and simurosertib in tumors collected at an intermediate time point from preclinical models treated in FIG. 5A and FIG. 5E. FIG. 6C: MYC mRNA levels in the tumors collected at endpoint in the prostate transformation models treated in FIG. 5A. FIG. 6D: Barplot showing proteasome activity in control and CDC7-inhibited (either pharmacologically by simurosertib, or genetically by CDC7 CRISPR KO) prostate transformation models. FIG. 6E: Western blots showing neuroendocrine marker expression in prostate models of transformation, when treated with enzalutamide, simurosertib or their combination. Specifically, for each treatment condition, isogenic control and MYC^{T58A}-expressing cells were analyzed.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach, Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning, Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

The present disclosure demonstrates that (1) CDC7 inhibition delays NE transformation in lung and prostate adenocarcinoma at high risk of transformation when treated with targeted therapy, and (2) CDC7 inhibition robustly sensitizes NE-transformed lung and prostate tumors to chemotherapy.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the term “adenocarcinoma” refers to cancer that forms in the glandular tissue, which lines certain internal organs and makes and releases substances in the body, such as mucus, digestive juices, and other fluids. Most cancers of the breast, lung, esophagus, stomach, colon, rectum, pancreas, prostate, and uterus are adenocarcinomas.

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), intratumorally, or topically. Administration includes self-administration and the administration by another.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of lung or prostate adenocarcinomas. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the terms “individual”, “patient”, or “subject” are used interchangeably and refer to an individual organism, a vertebrate, a mammal, or a human. In certain embodiments, the individual, patient or subject is a human.

As used herein, “prevention,” “prevent,” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

As used herein, a “sample” or “biological sample” refers to a body fluid or a tissue sample isolated from a subject. In some cases, a biological sample may consist of or comprise whole blood, platelets, red blood cells, white blood cells, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, endothelial cells, synovial fluid, lymphatic fluid, ascites fluid, interstitial or extracellular fluid and the like. The term “sample” may also encompass the fluid in spaces between cells, including gingival crevicular fluid, bone marrow, cerebrospinal fluid (CSF), saliva, mucus, sputum, semen, sweat, urine, or any other bodily fluids. Samples can be obtained from a subject by any means including, but not limited to, venipuncture, excretion, ejaculation, massage, biopsy, needle aspirate, lavage, scraping, surgical incision, or intervention or other means known in the art. A blood sample can be whole blood or any fraction thereof, including blood cells (red blood cells, white blood cells or leukocytes, and platelets), serum and plasma.

As used herein, the term “separate” therapeutic use refers to an administration of at least two active ingredients at the same time or at substantially the same time by different routes.

As used herein, the term “sequential” therapeutic use refers to administration of at least two active ingredients at different times. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the term “therapeutic agent” is intended to mean a compound that, when present in an effective amount, produces a desired therapeutic effect on a subject in need thereof.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

### Therapeutic Methods of the Present Technology

In one aspect, the present disclosure provides a method for treating or preventing neuroendocrine tumor formation in a patient diagnosed with adenocarcinoma comprising administering to the patient an effective amount of a CDC7 inhibitor, wherein the adenocarcinoma exhibits reduced expression and/or activity of TP53 and RB1. In some embodiments, the adenocarcinoma comprises (a) a genetic mutation, an indel, a copy number alteration or epigenetic downregulation in TP53 and (b) a genetic mutation, an indel, a copy number alteration or epigenetic downregulation in RB1.

In another aspect, the present disclosure provides a method for preventing neuroendocrine tumor formation in a patient diagnosed with adenocarcinoma comprising administering to the patient an effective amount of a CDC7 inhibitor and an effective amount of at least one chemotherapeutic drug, wherein the adenocarcinoma exhibits reduced expression and/or activity of TP53 and RB1. Also provided herein is a method for enhancing responsiveness of a patient with neuroendocrine tumors to systemic chemotherapy comprising administering to the patient an effective amount of a CDC7 inhibitor and an effective amount of at least one chemotherapeutic drug, wherein the neuroendocrine tumors exhibit reduced expression and/or activity of TP53 and RB1. In some embodiments, the adenocarcinoma or the neuroendocrine tumors comprise(s) (a) a genetic mutation, an indel, a copy number alteration or epigenetic downregulation in TP53 and (b) a genetic mutation, an indel, a copy number alteration or epigenetic downregulation in RB1. In some embodiments, the CDC7 inhibitor and the at least one chemotherapeutic drug are administered sequentially, simultaneously, or separately. Additionally or alternatively, in some embodiments, the at least one chemotherapeutic drug is administered orally, intranasally, parenterally, intravenously, intramuscularly, intraperitoneally, subcutaneously, intratumorally, topically, by inhalation spray, buccally, or via an implanted reservoir. The at least one chemotherapeutic drug may be an alkylating agent, a platinum agent, a taxane, a vinca agent, an aromatase inhibitor, a cytostatic alkaloid, a cytotoxic antibiotic, an antimetabolite, an endocrine/hormonal agent, or a bisphosphonate therapy agent. Examples of chemotherapeutic drugs include, but are not limited to cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, risedronate, pamidronate, ibandronate, alendronate, denosumab, zoledronate, tykerb, anthracyclines (e.g., daunorubicin and doxorubicin), oxaliplatin, melphalan, etoposide, mechlorethamine, bleomycin, microtubule poisons, annonaceous acetogenins, abraxane, leucovorin, nab-paclitaxel, everolimus, pegylated-hyaluronidase, pemetrexed, folinic acid, MK2206, GDC-0449, IPI-926, M402, LY293111 or combinations thereof.

In yet another aspect, the present disclosure provides a method for preventing neuroendocrine tumor formation in a patient diagnosed with adenocarcinoma comprising administering to the patient an effective amount of a CDC7 inhibitor and an effective amount of an androgen receptor (AR) inhibitor. The adenocarcinoma may exhibit reduced expression and/or activity of TP53 and RB1. In certain embodiments, the adenocarcinoma comprises (a) a genetic mutation, an indel, a copy number alteration or epigenetic downregulation in TP53 and (b) a genetic mutation, an indel, a copy number alteration or epigenetic downregulation in RB1. Additionally or alternatively, in some embodiments, the CDC7 inhibitor and the AR inhibitor are administered sequentially, simultaneously, or separately. Additionally or alternatively, in some embodiments, the AR inhibitor is administered orally, intranasally, parenterally, intravenously, intramuscularly, intraperitoneally, subcutaneously, intratumorally, topically, by inhalation spray, buccally, or via an implanted reservoir.

Examples of AR inhibitors include, but are not limited to apalutamide, bicalutamide, darolutamide, enzalutamide, flutamide, abiraterone acetate, ARN-509, and nilutamide.

In another aspect, the present disclosure provides a method for preventing neuroendocrine tumor formation in a patient diagnosed with adenocarcinoma comprising administering to the patient an effective amount of a CDC7 inhibitor and an effective amount of an epidermal growth factor receptor (EGFR) inhibitor (EGFRi) (e.g., EGFR tyrosine kinase inhibitor (TKI), anti-EGFR antibodies). The adenocarcinoma may exhibit reduced expression and/or activity of TP53 and RB1. In certain embodiments, the adenocarcinoma comprises (a) a genetic mutation, an indel, a copy number alteration or epigenetic downregulation in TP53 and (b) a genetic mutation, an indel, a copy number alteration or epigenetic downregulation in RB1. Additionally or alternatively, in some embodiments, the CDC7 inhibitor and the EGFRi are administered sequentially, simultaneously, or separately. Additionally or alternatively, in some embodiments, the EGFRi is administered orally, intranasally, parenterally, intravenously, intramuscularly, intraperitoneally, subcutaneously, intratumorally, topically, by inhalation spray, buccally, or via an implanted reservoir.

Examples of EGFRis include, but are not limited to, osimertinib, afatinib, erlotinib, gefitinib, icotinib, dacomitinib, rociletinib, olmutinib, cetuximab, panitumumab, nimotuzumab, and necitumumab.

In any and all embodiments of the methods disclosed herein, the patient is diagnosed with TP53⁻/⁻and RB1⁻/⁻mutant adenocarcinoma. In some embodiments, the TP53⁻/⁻and RB1⁻/⁻mutant adenocarcinoma is lung adenocarcinoma or prostate adenocarcinoma.

Additionally or alternatively, in some embodiments, the CDC7 inhibitor is administered orally, intranasally, parenterally, intravenously, intramuscularly, intraperitoneally, subcutaneously, intratumorally, topically, by inhalation spray, buccally, or via an implanted reservoir. Examples of CDC7 inhibitors include, but are not limited to simurosertib (TAK-931), PHA-767491, carvedilol, dequalinium chloride, ticagrelor, and clofoctol.

In any and all embodiments of the methods disclosed herein, the patient is human. Additionally or alternatively, in some embodiments, the patient is non-responsive to at least one prior line of cancer therapy such as chemotherapy.

Additionally or alternatively, in some embodiments of the methods disclosed herein, the CDC7 inhibitor can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), simultaneously with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the AR inhibitor, EGFRi, or chemotherapeutic drug to the patient.

In some embodiments, the CDC7 inhibitor and the AR inhibitor or EGFRi or chemotherapeutic drug are administered to a patient, for example, a mammal, such as a human, in a sequence and within a time interval such that the inhibitor or drug that is administered first acts together with the inhibitor or drug that is administered second to provide greater benefit than if each inhibitor were administered alone.

For example, the CDC7 inhibitor and the AR inhibitor or EGFRi or chemotherapeutic drug can be administered at the same time or sequentially in any order at different points in time; however, if not administered at the same time, the CDC7 inhibitor and the AR inhibitor or EGFRi or chemotherapeutic drug are administered sufficiently close in time so as to provide the desired therapeutic or prophylactic effect of the combination of the two inhibitors. In one embodiment, the CDC7 inhibitor and the AR inhibitor or EGFRi or chemotherapeutic drug exert their effects at times which overlap. In some embodiments, the CDC7 inhibitor and the AR inhibitor or EGFRi or chemotherapeutic drug are each administered as separate dosage forms, in any appropriate form and by any suitable route. In other embodiments, the CDC7 inhibitor and the AR inhibitor or EGFRi or chemotherapeutic drug are administered simultaneously in a single dosage form.

It will be appreciated that the frequency with which any of these therapeutic agents can be administered once or more than once over a period of about 2 days, about 3 days, about 4 days, about 5 days, about 6 days, about 7 days, about 8 days, about 9 days, about 10 days, about 11 days, about 12 days, about 13 days, about 14 days, about 20 days, about 28 days, about a week, about 2 weeks, about 3 weeks, about 4 weeks, about a month, about every 2 months, about every 3 months, about every 4 months, about every 5 months, about every 6 months, about every 7 months, about every 8 months, about every 9 months, about every 10 months, about every 11 months, about every year, about every 2 years, about every 3 years, about every 4 years, or about every 5 years.

For example, the CDC7 inhibitor or the AR inhibitor or EGFRi or chemotherapeutic drug may be administered daily, weekly, biweekly, or monthly for a particular period of time. The CDC7 inhibitor or the AR inhibitor or EGFRi or chemotherapeutic drug may be dosed daily over a 14 day time period, or twice daily over a seven day time period. The CDC7 inhibitor or AR inhibitor or EGFRi or chemotherapeutic drug may be administered daily for 7 days.

Alternatively, a CDC7 inhibitor or AR inhibitor or EGFRi or chemotherapeutic drug may be administered daily, weekly, biweekly, or monthly for a particular period of time followed by a particular period of non-treatment. In some embodiments, the CDC7 inhibitor or AR inhibitor or EGFRi or chemotherapeutic drug can be administered daily for 14 days followed by seven days of non-treatment, and repeated for two more cycles of daily administration for 14 days followed by seven days of non-treatment. In some embodiments, the CDC7 inhibitor or AR inhibitor or EGFRi or chemotherapeutic drug can be administered twice daily for seven days followed by 14 days of non-treatment, which may be repeated for one or two more cycles of twice daily administration for seven days followed by 14 days of non-treatment.

In some embodiments, the CDC7 inhibitor or AR inhibitor or EGFRi or chemotherapeutic drug is administered daily over a period of 14 days. In another embodiment, the CDC7 inhibitor or AR inhibitor or EGFRi or chemotherapeutic drug is administered daily over a period of 12 days, or 11 days, or 10 days, or nine days, or eight days. In another embodiment, the CDC7 inhibitor or AR inhibitor or EGFRi or chemotherapeutic drug is administered daily over a period of seven days. In another embodiment, the CDC7 inhibitor or AR inhibitor or EGFRi or chemotherapeutic drug is administered daily over a period of six days, or five days, or four days, or three days.

In some embodiments, individual doses of the CDC7 inhibitor and the AR inhibitor or EGFRi or chemotherapeutic drug are administered within a time interval such that the two therapeutic agents can work together (e.g., within 1 hour, 2 hours, 4 hours, 6 hours, 12 hours, 24 hours, 48 hours, 72 hours, 96 hours, 5 days, 6 days, 1 week, or 2 weeks). In some embodiments, the treatment period during which the therapeutic agents are administered is then followed by a non-treatment period of a particular time duration, during which the therapeutic agents are not administered to the patient. This non-treatment period can then be followed by a series of subsequent treatment and non-treatment periods of the same or different frequencies for the same or different lengths of time. In some embodiments, the treatment and non-treatment periods are alternated. It will be understood that the period of treatment in cycling therapy may continue until the patient has achieved a complete response or a partial response, at which point the treatment may be stopped.

Alternatively, the period of treatment in cycling therapy may continue until the patient has achieved a complete response or a partial response, at which point the period of treatment may continue for a particular number of cycles. In some embodiments, the length of the period of treatment may be a particular number of cycles, regardless of patient response. In some other embodiments, the length of the period of treatment may continue until the patient relapses.

In some embodiments, the CDC7 inhibitor and the AR inhibitor or EGFRi or chemotherapeutic drug are each administered at a dose and schedule typically used for that agent during monotherapy. In other embodiments, when the CDC7 inhibitor and the AR inhibitor or EGFRi or chemotherapeutic drug are administered concomitantly, one or both of the agents can advantageously be administered at a lower dose than typically administered when the agent is used during monotherapy, such that the dose falls below the threshold that an adverse side effect is elicited.

The therapeutically effective amounts or suitable dosages of the CDC7 inhibitor and the AR inhibitor or EGFRi or chemotherapeutic drug in combination depends upon a number of factors, including the nature of the severity of the condition to be treated, the particular inhibitor, the route of administration and the age, weight, general health, and response of the individual patient. In certain embodiments, the suitable dose level is one that achieves a therapeutic response as measured by tumor regression or other standard measures of disease progression, progression free survival, or overall survival. In other embodiments, the suitable dose level is one that achieves this therapeutic response and also minimizes any side effects associated with the administration of the therapeutic agent.

Suitable daily dosages of AR inhibitors can generally range, in single or divided or multiple doses, from about 10% to about 120% of the maximum tolerated dose as a single agent. In certain embodiments, the suitable dosages of AR inhibitors are from about 20% to about 100% of the maximum tolerated dose as a single agent. In other embodiments, the suitable dosages of AR inhibitors are from about 25% to about 90% of the maximum tolerated dose as a single agent. In some embodiments, the suitable dosages of AR inhibitors are from about 30% to about 80% of the maximum tolerated dose as a single agent. In other embodiments, the suitable dosages of AR inhibitors are from about 40% to about 75% of the maximum tolerated dose as a single agent. In some embodiments, the suitable dosages of AR inhibitors are from about 45% to about 60% of the maximum tolerated dose as a single agent. In other embodiments, suitable dosages of AR inhibitors are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Suitable daily dosages of EGFRis can generally range, in single or divided or multiple doses, from about 10% to about 120% of the maximum tolerated dose as a single agent. In certain embodiments, the suitable dosages of EGFRis are from about 20% to about 100% of the maximum tolerated dose as a single agent. In other embodiments, the suitable dosages of EGFRis are from about 25% to about 90% of the maximum tolerated dose as a single agent. In some embodiments, the suitable dosages of EGFRis are from about 30% to about 80% of the maximum tolerated dose as a single agent. In other embodiments, the suitable dosages of EGFRis are from about 40% to about 75% of the maximum tolerated dose as a single agent. In some embodiments, the suitable dosages of EGFRis are from about 45% to about 60% of the maximum tolerated dose as a single agent. In other embodiments, suitable dosages of EGFRis are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Suitable daily dosages of chemotherapeutic drugs can generally range, in single or divided or multiple doses, from about 10% to about 120% of the maximum tolerated dose as a single agent. In certain embodiments, the suitable dosages of chemotherapeutic drugs are from about 20% to about 100% of the maximum tolerated dose as a single agent. In other embodiments, the suitable dosages of chemotherapeutic drugs are from about 25% to about 90% of the maximum tolerated dose as a single agent. In some embodiments, the suitable dosages of chemotherapeutic drugs are from about 30% to about 80% of the maximum tolerated dose as a single agent. In other embodiments, the suitable dosages of chemotherapeutic drugs are from about 40% to about 75% of the maximum tolerated dose as a single agent. In some embodiments, the suitable dosages of chemotherapeutic drugs are from about 45% to about 60% of the maximum tolerated dose as a single agent. In other embodiments, suitable dosages of chemotherapeutic drugs are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Suitable daily dosages of CDC7 inhibitors can generally range, in single or divided or multiple doses, from about 10% to about 120% of the maximum tolerated dose as a single agent. In certain embodiments, the suitable dosages of CDC7 inhibitors are from about 20% to about 100% of the maximum tolerated dose as a single agent. In some other embodiments, the suitable dosages of CDC7 inhibitors are from about 25% to about 90% of the maximum tolerated dose as a single agent. In some other embodiments, the suitable dosages of CDC7 inhibitors are from about 30% to about 80% of the maximum tolerated dose as a single agent. In some other embodiments, the suitable dosages of CDC7 inhibitors are from about 40% to about 75% of the maximum tolerated dose as a single agent. In some other embodiments, the suitable dosages of CDC7 inhibitors are from about 45% to about 60% of the maximum tolerated dose as a single agent. In other embodiments, suitable dosages of CDC7 inhibitors are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the CDC7 inhibitor or AR inhibitor or EGFRi or chemotherapeutic drug, sufficient for achieving a therapeutic or prophylactic effect, may range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of CDC7 inhibitor or AR inhibitor or EGFRi or chemotherapeutic drug ranges from 0.001-10,000 micrograms per kg body weight. In one embodiment, CDC7 inhibitor or AR inhibitor or EGFRi or chemotherapeutic drug concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of a CDC7 inhibitor or AR inhibitor or EGFRi or chemotherapeutic drug may be defined as a concentration of the CDC7 inhibitor or AR inhibitor or EGFRi or chemotherapeutic drug at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Formulations Including the CDC7 Inhibitors of the Present Technology

The pharmaceutical compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human. Formulations including any CDC7 inhibitor disclosed herein may be designed to be short-acting, fast-releasing, or long-acting. In other embodiments, compounds can be administered in a local rather than systemic means, such as administration (e.g., by injection) at a tumor site.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

### Kits

The present disclosure provides kits comprising one or more CDC7 inhibitors disclosed herein, and instructions for treating or preventing neuroendocrine tumor formation. When simultaneous administration is contemplated, the kit may comprise a CDC7 inhibitor and an AR inhibitor or EGFRi or chemotherapeutic drug that has been formulated into a single pharmaceutical composition such as a tablet, or as separate pharmaceutical compositions. When the CDC7 inhibitor and the AR inhibitor or EGFRi or chemotherapeutic drug are not administered simultaneously, the kit may comprise a CDC7 inhibitor and an AR inhibitor or EGFRi or chemotherapeutic drug that has been formulated as separate pharmaceutical compositions either in a single package, or in separate packages.

Additionally or alternatively, in some embodiments, the kits further comprise at least one AR inhibitor that are useful for treating or preventing neuroendocrine tumor formation. Examples of AR inhibitors include, but are not limited to apalutamide, bicalutamide, darolutamide, enzalutamide, flutamide, abiraterone acetate, ARN-509, and nilutamide.

Additionally or alternatively, in some embodiments, the kits further comprise at least one EGFRi that are useful for treating treating or preventing neuroendocrine tumor formation. Examples of EGFRis include, but are not limited to, osimertinib, afatinib, erlotinib, gefitinib, icotinib, dacomitinib, rociletinib, olmutinib, cetuximab, panitumumab, nimotuzumab, and necitumumab.

Additionally or alternatively, in some embodiments, the kits further comprise at least one chemotherapeutic agent that are useful for treating or preventing neuroendocrine tumor formation. Examples of chemotherapeutic drugs include, but are not limited to cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, risedronate, pamidronate, ibandronate, alendronate, denosumab, zoledronate, tykerb, anthracyclines (e.g., daunorubicin and doxorubicin), oxaliplatin, melphalan, etoposide, mechlorethamine, bleomycin, microtubule poisons, annonaceous acetogenins, abraxane, leucovorin, nab-paclitaxel, everolimus, pegylated-hyaluronidase, pemetrexed, folinic acid, MK2206, GDC-0449, IPI-926, M402, LY293111 or combinations thereof.

The kits may further comprise pharmaceutically acceptable excipients, diluents, or carriers that are compatible with one or more kit components described herein. Optionally, the above described components of the kits of the present technology are packed in suitable containers and labeled for the treatment or prevention of neuroendocrine tumors. The kits may optionally include instructions customarily included in commercial packages of therapeutic products, that contain information about, for example, the indications, usage, dosage, manufacture, administration, contraindications and/or warnings concerning the use of such therapeutic products.

### EXAMPLES

The present technology is further illustrated by the following Examples, which should not be construed as limiting in any way. For each of the examples below, any MEK inhibitor or CDK4/6 inhibitor described herein could be used.

### Example 1: General Methods and Procedures

### Cell Lines

H1563 (CRL-5875), H660 (CRL-5813), H69 (HTB-119), H82 (HTB-175), SHP-77 (CRL-2195), H526 (CRL-5811), H446 (HTB-171), H146 (HTB-173) and DMS-114 (CRL-2066) were purchased from ATCC. H1563 (CRL-5875) and H660 (CRL-5813) were purchased from ATCC. LnCap and 22PC cell lines were kindly shared by Sawyers lab at MSKCC and maintained as previously described¹⁶. Cell lines were authenticated through the STR characterization method and regularly tested for Mycoplasma (Universal Mycoplasma Detection Kit, #30-1012K, ATCC). All experiments were performed in low passage cells. All cell lines were cultured according to ATCC guidelines or as previously described¹⁶.

### Plasmid Vectors and Transductions

To generate Cas9-expressing cell lines, cells were spin-transduced with lentiviral particles made out of a lentiviral plasmid designed to constitutively express Cas9 (#125592, Addgene) as described in ¹⁷, and selected with blasticidin 2.5 μg/mL.

Cells were similarly spin-transduced as described in ¹⁷ with lentiviral particles made out of lentiviral LV03 vectors expressing sgRNAs for CDC7 (#HSPD0000047627 and HSPD0000047628, Sigma) or the respective control vector expressing a safe targeting sgRNA BFP (#HSCONTROL_AAVS1 on LV03, Sigma), or the Lv151 vector overexpressing CDC7 (#EX-M0793-Lv151 Genecopoeia).

TP53/RB1-deficient PRAD cell lines were generated as previously described¹⁶. TP53/RB1-deficient LUAD cell lines were generated by lentiviral transduction of a construct expressing a dominant negative TP53 isoform and a short hairpin RNA against RB1, produced from the FU-CYW vector that was previously described¹⁸ and kindly shared by Dr. Owen Witte.

### Monotherapy Cytotoxicity Assay

Cytotoxic assays were performed as described in ¹⁹. A total of 1,500 cells/well were seeded in 96-well plates and treated with the drugs/doses described for 96 hours. Viability was assessed with the CellTiter-Glo 2.0 Assay (Promega, G9242) as indicated by manufacturer.

### Synergy Assays

Cells were seeded in 96-well plates (1500 cells/well) and treated with the interval of concentrations of cisplatin or simurosertib for 5 days. Then, cell viability was assessed with CellTiter-Glo 2.0 Assay (Promega, G9242) and normalized to the untreated wells. Synergy was calculated using the ZIP method using the SynergyFinder web application (2.0)²⁰.

### Immunoblot

Protein extraction and western blot were performed as previously described²¹. Antibodies for CDC7 (#3603, Cell Signaling Technology), MYC (#5605, Cell Signaling Technology), synaptophysin (#36406, Cell Signaling Technology), CD56 (#99746, Cell Signaling Technology), AR (#5153, Cell Signaling Technology vinculin (#13901, Cell Signaling Technology), tubulin (#3873, Cell Signaling Technology) and actin (#3700, Cell Signaling Technology). Quantifications were performed with the Image Studio software (Version 3.1, Li-Cor). Immunohistochemistry for CDC7 was performed with the CDC7 antibody #MA5-12589 from Invitrogen in TMAs including resected tumor samples form SCLC and NSCLC patients. All study subjects had provided signed informed consent for biospecimen analyses under an Institutional Review Board-approved protocol.

### Propidium Iodide/Annexin V Assays

Parental cells were treated with cisplatin (GI20), selinexor (GI20) or their combination for 3 days. Alternatively, control and XPO1 CRISPR-KO cells were treated with cisplatin (GI20) for 3 days. Then, cells were collected and stained with the APC Annexin V Apoptosis Detection Kit with PI (#640932, Biolegend) and apoptosis was analyzed by flow cytometry as previously described²²

Data from Cancer Cell Line Encyclopedia

CDC7 mRNA expression data from Cancer Cell Line Encyclopedia (CCLE)¹¹ was downloaded from UCSC Xenabrowser portal (https://xenabrowser.net/) in December 2020.

### In Vivo Treatments

5-10 female 6-week old NOD. Cg-Prkdc<scid>Il2rg<tm1Wjl>/SzJ (NSG) mice (PDXs) or female 6-week-old athymic nude mice (cell line xenografts) were engrafted per treatment arm and until tumors reached 100-150 mm³. At that point, mice were randomized into groups and treated with either vehicle, cisplatin (2 mg/kg i.p. once/week), etoposide (3 mg/kg i.p. QDx3), simurosertib (40 mg/kg p.o. QDx3), enzalutamide (10 mg/kg p.o. QDx5), osimertinib (25 mg/kg p.o. QDx5) or the combinations of cisplatin+etoposide, cisplatin+simurosertib, enzalutamide+simurosertib or osimertinib+simurosertib at the previously mentioned doses. Mice weights and tumor volumes were measured twice a week and mice were sacrificed when tumors reached humane endpoint. All animal experiments were approved by the Memorial Sloan Kettering Cancer Center (MSKCC) Animal Care and Use Committee.

### RNA Extraction

Frozen tissues or cell pellets were weighed and homogenized in RLT and nucleic acids were extracted using the AllPrep DNA/RNA Mini Kit (QIAGEN, #80204) according to the manufacturer's instructions. RNA was eluted in nuclease-free water.

### RNA Extraction

Frozen tissues or cell pellets were weighed and homogenized in RLT and nucleic acids were extracted using the AllPrep DNA/RNA Mini Kit (QIAGEN, #80204) according to the manufacturer's instructions. RNA was eluted in nuclease-free water.

### RNAseq Alignment and Quantification

Transcript abundances were quantified using RNA-seq reads by Salmon v1.1.0²³ Raw reads of RNA-seq were mapped to 25 mer indexed hg38 genome. In addition to default settings, mapping validation (—validatemappings), bootstrapping with 30 re-samplings (—numBootstraps), sequence specific biases correction (—seqBias), coverage biases correction (—posBias) and GC biases correction (—gcBias) were also enabled. Transcripts were mapped to genes based on Ensembl 92²⁴, normalized by size factor at gene level. Subsequently the differential gene expression were evaluated on Salmon output files using Sleuth v0.30.0²⁵ in gene mode. Wald test was performed on differential gene expressions. Genes were marked as significantly differentially expressed if the False Discovery Rates, q, calculated using the Benjamini-Hochberg method, was less than 0.05, and beta (Sleuth-based estimation of log 2 fold change)>0.58, which approximately equivalent to a log 2 fold change of 1.5.

### Publicly Available RNAseq Datasets Analyses

The public sets were divided into four groups according to their mutation status of TP53 and RB1, as the following, TP53WT/RB1WT, TP53MT/RB1WT, TP53WT/RB1MT, and TP53MT/RB1MT. RNAseq expression distribution of XPO1, SOX2 and CDC7 were presented in box plots for the above four groups of samples. RNAseq expression values were downloaded through cBioPortal. <data type 1:RSEM> The expression levels for LUSD (OncoSG, Nat Genet 2020) are in RSEM (RNAseq by Expectation-Maximization) that have been normalized using DESeq2 v.1.16.1 followed by log transformation while that for PRAD (TCGA, PanCancer) are in batch normalized RSEM then followed by log transformation. <data type 2: RSEM z-score> Log-transformed mRNA expression z-scores compared to the expression distribution of all samples were downloaded for both LUSD (OncoSG, Nat Genet 2020)²⁶ and PRAD (TCGA, PanCancer)²⁷. The pairwise comparisons of mean expressions were conducted among previously mentioned four groups and evaluated by Wilcoxon test. (Using traditional RNAseq DEG approach to evaluate DE p value by limma pipeline: linear modelling was applied on the normalized and log transformed RSEM values which are assumed to be normally distributed using limma (v3.28.14)²⁸. The coefficients and standard errors were then estimated for each pair of contrast from the linear model. Empirical Bayes Statistics for differential expressions were carried out to evaluate the significance level.)

The expression values of XPO1 and SOX2 were correlated in scatter plots for previously mention seven cohorts. RNAseq expression values were downloaded through cBioPortal. <data type 1:RSEM> The expression levels are in RSEM (RNAseq by Expectation-Maximization) that have been using DESeq2 v.1.16.1 normalization, LUSD (OncoSG, Nat Genet 2020)²⁶, or batch normalized followed by log transformation. <data type 2: RSEM z-score> Log-transformed mRNA expression z-scores compared to the expression distribution of all samples were downloaded. The expression correlations were evaluated by Pearson (Spearman).

### Pathway Enrichment Analyses

Gene set enrichment analysis (GSEA)²⁹ was conducted on the full sets of differential gene expression output from the previously mentioned comparisons. Genes were ranked by p value scores computed as −log10(p value)*(sign of beta). The annotations of gene set were taken from Molecular Signatures Database (MSigDB v7.0.1)^{29,30} of gene set enrichment was evaluated using permutation test and the p value was adjusted by Benjamini-Hochberg procedure. Any enriched gene sets with adjusted p value≤0.1 were regarded as significant. This analysis was conducted using ClusterProfiler R package v3.18.1³¹. Some enriched gene sets of interests were selected and their pathway annotations were concatenated manually to remove redundancy and achieve high level generality. When the pathway terms were merged, median enrichment score was taken as the new group enrichment score, p values were aggregated using Fisher's method from the Aggregation R package³², and core enrichment of genes were collapsed. The consolidated gene sets enrichment were then presented in dot plots.

### ATAC-seq

The reads were trimmed for both quality and Illumina adaptor sequences using trim_galore v0.4.4 (https://github.com/FelixKrueger/TrimGalore) in the pair-end mode. Then the raw reads were aligned to human assembly hg38 using bowtie2 v2.3.4³³ using the default parameters. Aligned reads with the same start site and orientation were removed using the Picard tool (https://broadinstitute.github.io/picard/). Enriched regions in individual samples were called using MACS2³⁴ and then filtered against genomic ‘blacklisted’ regions (http://mitra.stanford.edu/kundaje/akundaje/release/blacklists/hg38-human/hg38.blacklist.bed.gz). The filtered peaks within 500 bp were merged to create an union of peak atlas. Raw read counts were tabulated over this peak atlas using featureCounts v1.6.0³⁵. The read counts were then normalized with DESeq2. The read density profile in the format of bigwig file for each sample was created using the BEDTools suite (https://bedtools.readthedocs.io) with the normalization factor from DESeq2³⁶. All bigwig genome tracks on XPO1 gene region were generated using pyGenomeTracks v3.5³⁷.

### Clinical Samples

All study subjects had provided signed informed consent for biospecimen analyses under an Institutional Review Board-approved protocol.

### Proteasome Activity Assay

Proteasome activity was measured with the Proteasome 20S Activity Assay Kit (#MAK172, Sigma), following the manufacturer's instructions.

### Example 2: CDC7 is Highly Expressed in SCLC and Exerts Pro-Oncogenic Effects

SCLCs are highly proliferative tumors exhibiting high dependency for cell cycle and DNA repair genes. Genes in these pathways are even upregulated during the process of adenocarcinoma to SCLC transformation^{1,2}, highlighting their importance in the SCLC setting, and consistently, targeting genes in these pathways such as CDK7³ or Chk1⁴ has yielded promising preclinical efficacy against SCLC tumors. The MCM complex, involved in the initiation of DNA replication before cell division, has been previously involved in chemotherapy resistance in the SCLC setting⁵. This complex is activated by CDC7⁶, a gene previously involved in tumorigenesis in different tumor settings⁷. CDC7 expression is induced by inactivation of TP53 and RB1-hallmarks of SCLC-in different tumor types, including lung cancer^{8,9}. These results were suggestive of a potential pro-oncogenic role for this gene in the SCLC setting. Additionally, the recent development of potent and clinically safe inhibitors for CDC7^{7,10} made it an attractive therapeutic target candidate for SCLC, and thus the role of CDC7 in this setting was evaluated.

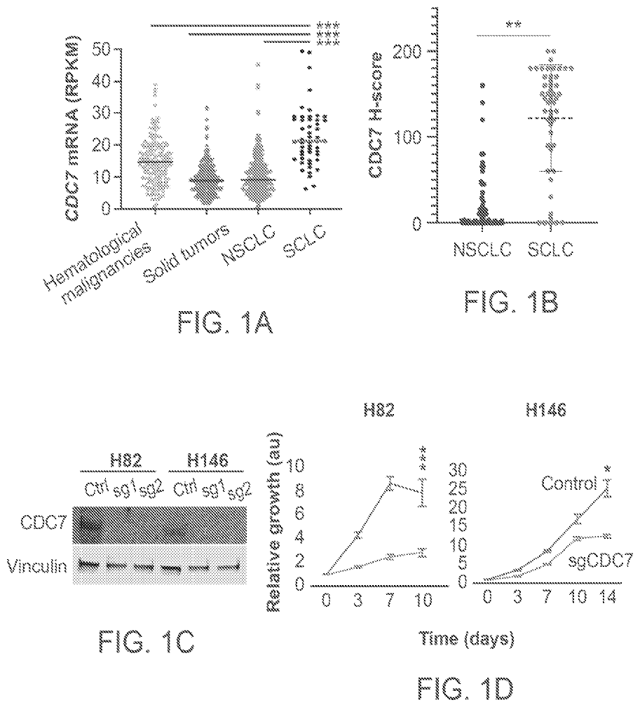

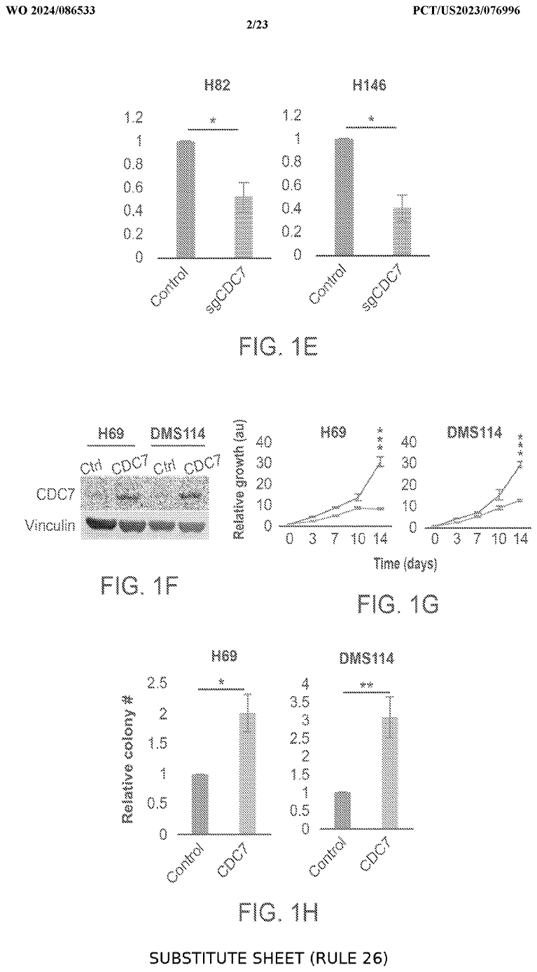

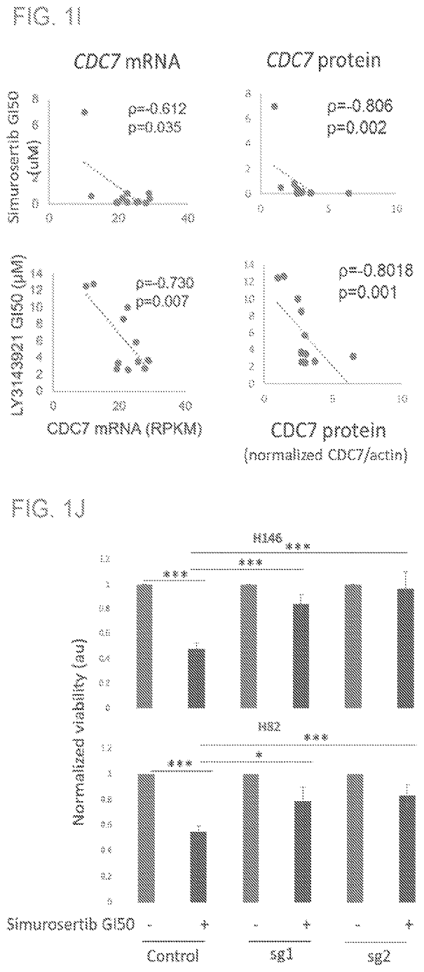

Assessment of CDC7 mRNA expression in cell lines derived from different tumor types obtained from the Cancer Cell Line Encyclopedia (CCLE)¹¹ suggested that CDC7 expression was higher in SCLC than in any other tumor type, and superior to that observed in non-small cell lung tumors (NSCLC, FIG. 1A). This finding was validated in clinical specimens by immunohistochemistry, which showed that SCLC tumors showed dramatically higher CDC7 protein expression as compared to NSCLC (FIG. 1B). To study if CDC7 is able to exert oncogenic effects in the SCLC setting, isogenic SCLC cell lines were generated with variable CDC7 expression. CRISPR-Cas9 knock out of CDC7 in two SCLC cell lines from the major SCLC subtypes (H82, SCLC-N and H146, SCLC-A) with high endogenous CDC7 expression (FIG. 1C) reduced proliferation (FIG. 1D) and colony formation (FIG. 1E) in these cell lines and consistently, ectopic overexpression of CDC7 in two cell lines with low endogenous CDC7 expression (H69, SCLC-A and DMS114, SCLC-Y, FIG. 1F), exerted the opposite effects (FIGS. 1G-1H), suggesting that CDC7 expression plays a pro-oncogenic role in the SCLC setting.

### Example 3: Inhibition of CDC7 Strongly Sensitizes SCLC PDXs to First- and Second-Line Chemotherapy

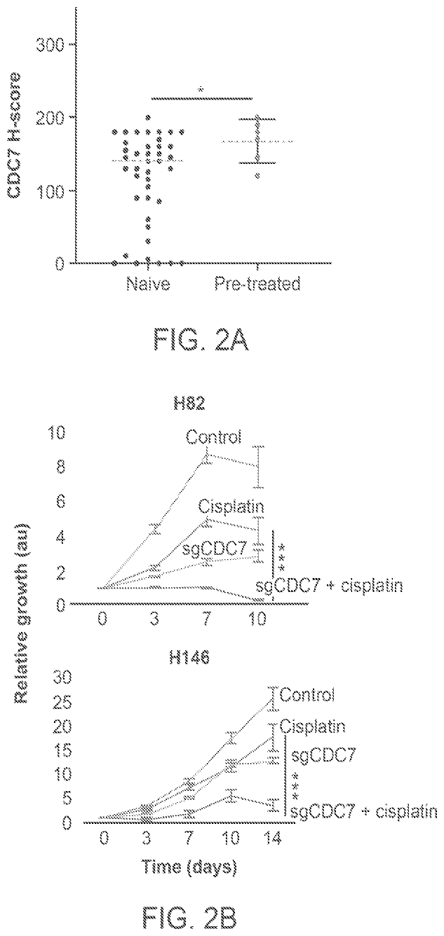

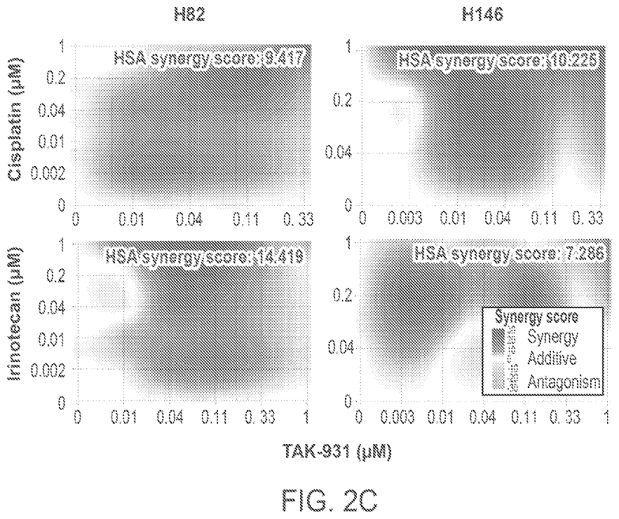

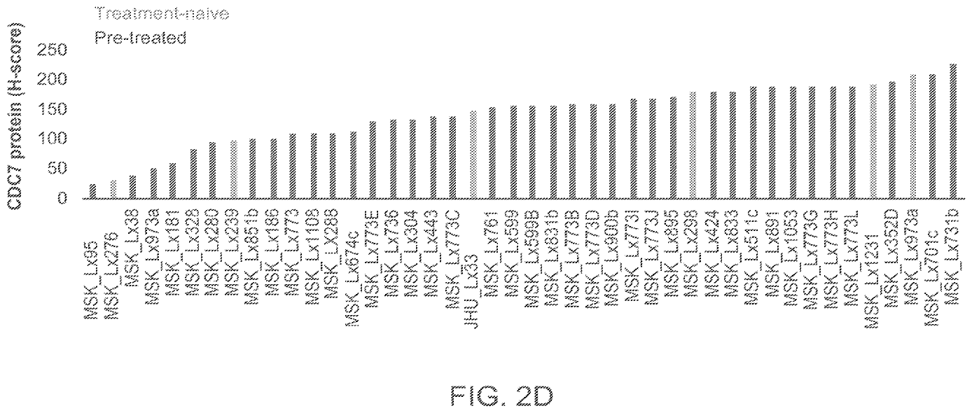

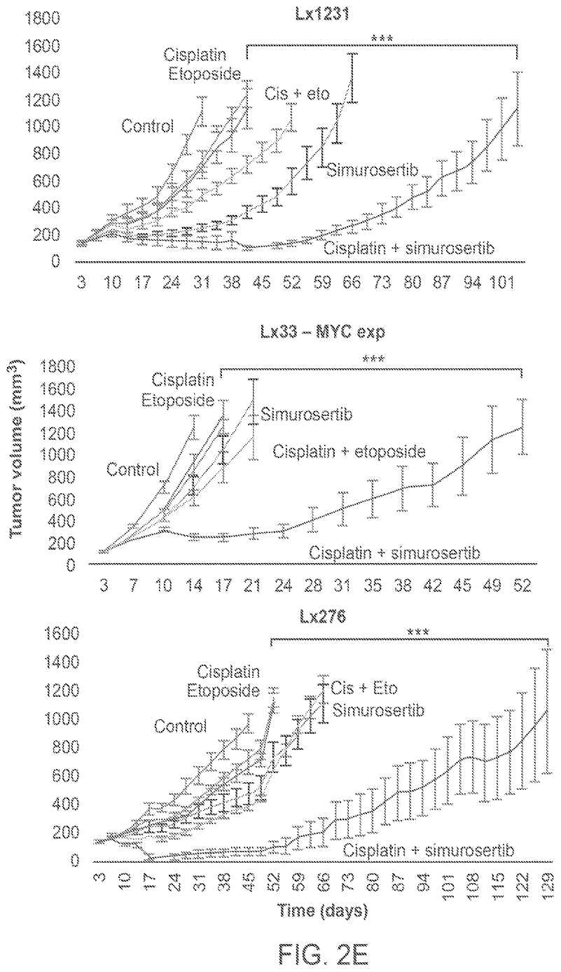

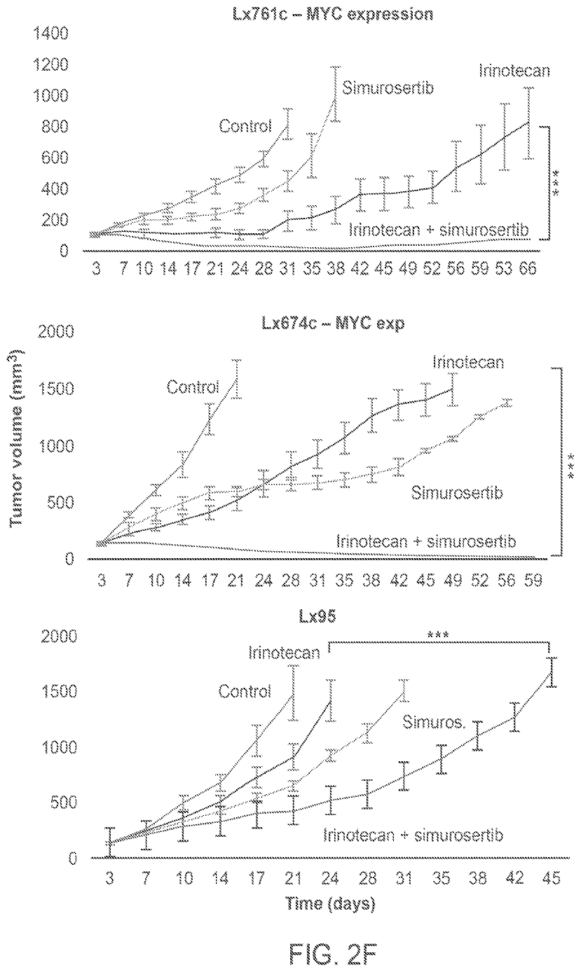

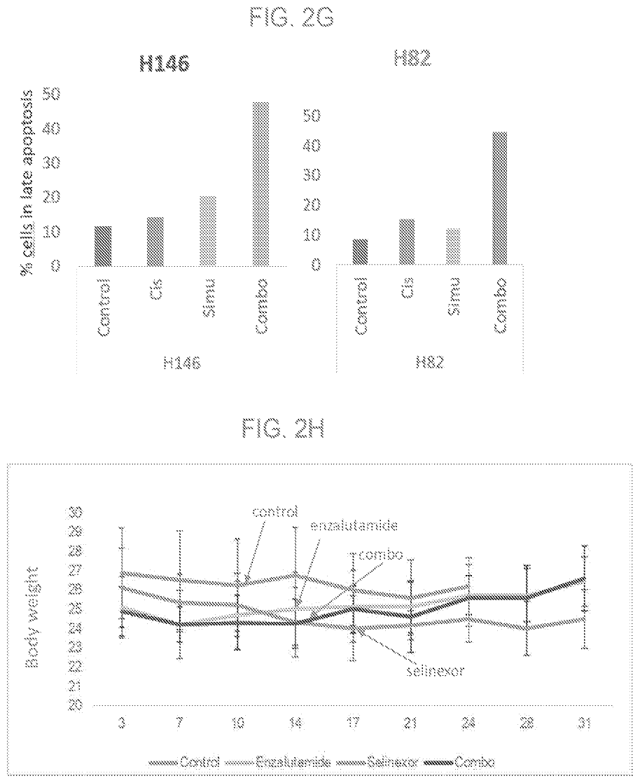

CDC7 inhibition has shown to sensitize to an array of chemotherapeutic agents in a variety of tumor types¹⁰; and the MCM complex, downstream CDC7, has been associated to chemotherapy resistance in SCLC⁵. Consistently, increased CDC7 protein expression was observed in SCLC tumors collected after prior chemotherapy treatment as compared to treatment-naïve specimens (FIG. 2A). Thus, the therapeutic potential of CDC7 inhibition in SCLC was examined. Initially, the sensitivity of an array of SCLC cell lines belonging to all SCLC subtypes to two different CDC7 inhibitors, simurosertib and LY3143921, was assessed to confirm their specificity in the SCLC setting (FIG. 1I). Sensitivity to either inhibitor was correlated with CDC7 mRNA and protein expression, suggesting that effects in terms of viability reduction by either inhibitor were specific in the SCLC setting. Then, due to its further clinical progress, simurosertib was selected for further experimentation. Assessment of simurosertib sensitivity in two SCLC cell lines revealed that CDC7 KO rendered the cells resistant to simurosertib, further supporting the rather on-target effects of this inhibitor (FIG. 1J).

Then, the role of CDC7 in SCLC chemosensitivity was assessed. The effect of CDC7 KO was studied in two high CDC7-expressing SCLC cell lines treated with cisplatin, an agent used in the first line setting for the treatment of SCLC (FIG. 2B). CDC7 KO induced a strong sensitivity to cisplatin in both cell lines (FIG. 2B), which was validated at the pharmacological level in apoptosis assays revealing increased apoptosis in cells treated with the combination of cisplatin and simurosertib than in cells treated with either inhibitor alone (FIG. 2G); and in in vitro synergy assays exhibiting high synergy between simurosertib and cisplatin as well as irinotecan, a chemotherapeutic agent used in the second line treatment of SCLC, again in both cell lines (FIG. 2C).

In the light of these results, the efficacy of simurosertib in combination with cisplatin and irinotecan was tested in SCLC PDXs derived from treatment-naïve and chemotherapy-relapsed SCLC tumors, respectively. Due to the CDC7 expression-dependent response to CDC7 inhibition (FIGS. 1J-1K) and to the dispersion of CDC7 protein expression in SCLC clinical specimens (FIG. 1B), we assessed whether the presence of high CDC7 expression levels is determinant to response to the combination of simurosertib and chemotherapy. To this aim, PDXs were treated with high (Lx1231, SCLC-A and Lx761c, SCLC-N), intermediate (Lx33, SCLC, N and Lx674c, SCLC-A) and low (Lx95, SCLC-A and Lx276, SCLC-A) CDC7 expression, to assess if CDC7 levels were predictive of the efficacy of the combination of simurosertib and chemotherapy (FIG. 2D). The treatment-naïve PDXs (FIG. 2E) exhibited exquisite sensitivity to the combination of cisplatin and simurosertib, dramatically outperforming the combination of cisplatin and etoposide. In the same line, in the PDXs derived from chemotherapy-relapsed tumors (FIG. 2F) simurosertib was able to strongly sensitize either model to irinotecan, including 3/5 and 5/5 complete tumor regressions in the Lx761c and Lx674c, respectively. In these 3 PDX models, the combination therapy showed superiority to irinotecan, including the low CDC7-expressing PDX model Lx95 (FIG. 2F), suggesting that the combination of simurosertib and irinotecan might be effective independently of the CDC7 expression levels. Altogether, these results nominate CDC7 as a therapeutic target in SCLC and potent sensitizer to first and second line therapies in current use.

### Example 4: CDC7 is Upregulated during NE Transformation and CDC7 Inhibition Sensitizes NE-Transformed Tumors to Chemotherapy

The oncogenic role for CDC7 in SCLC (FIGS. 1C-1H) and the superior CDC7 levels in this setting versus other tumor types and particularly LUAD (FIGS. 1A-1B) was suggestive of a key role for CDC7 in SCLC and prompted the study of a potential role for CDC7 in adenocarcinoma to NE transformation.

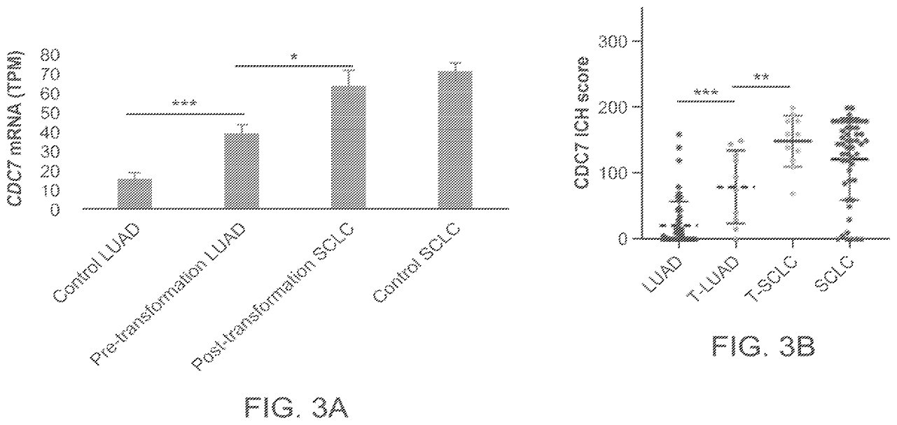

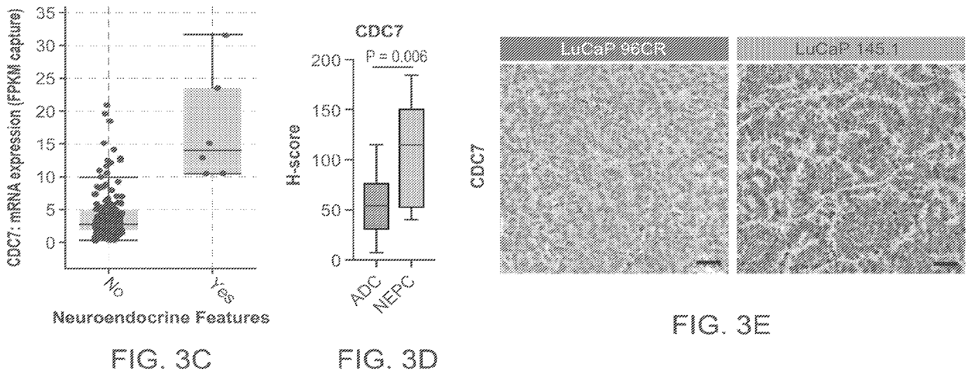

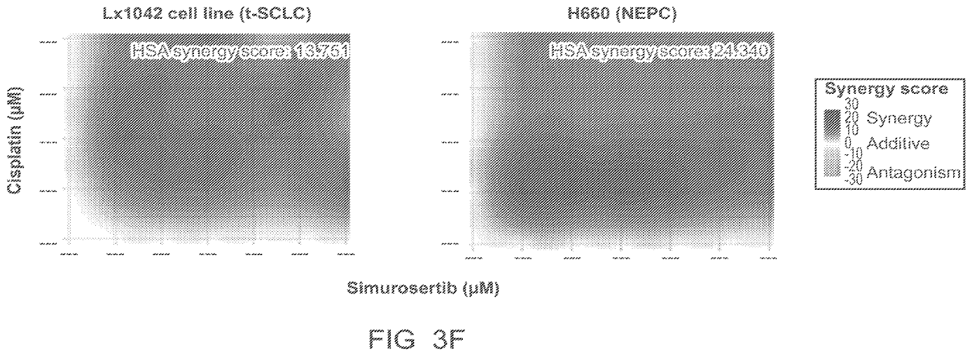

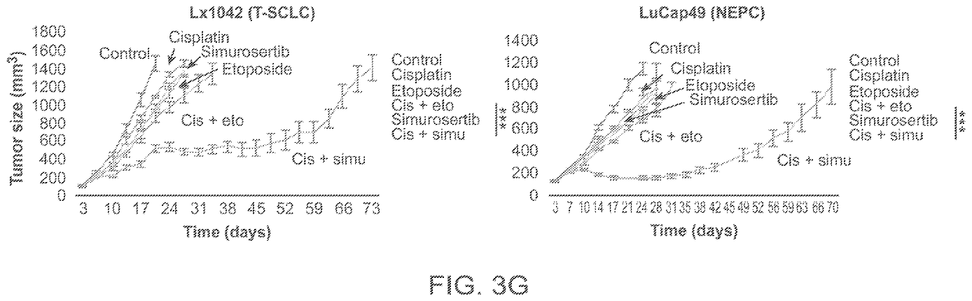

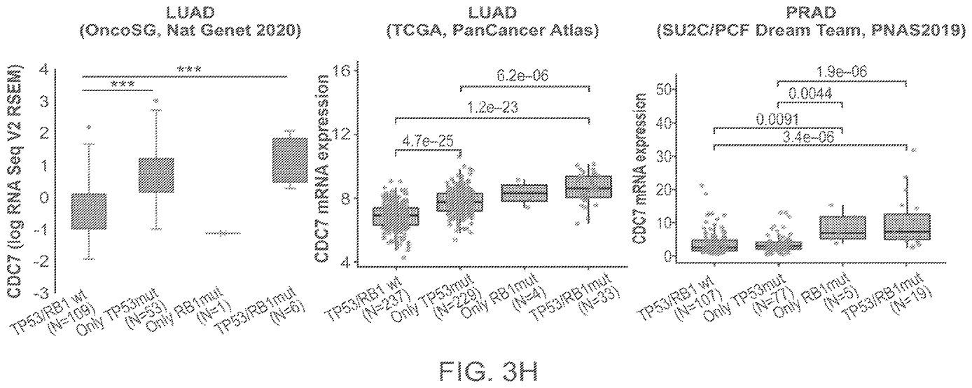

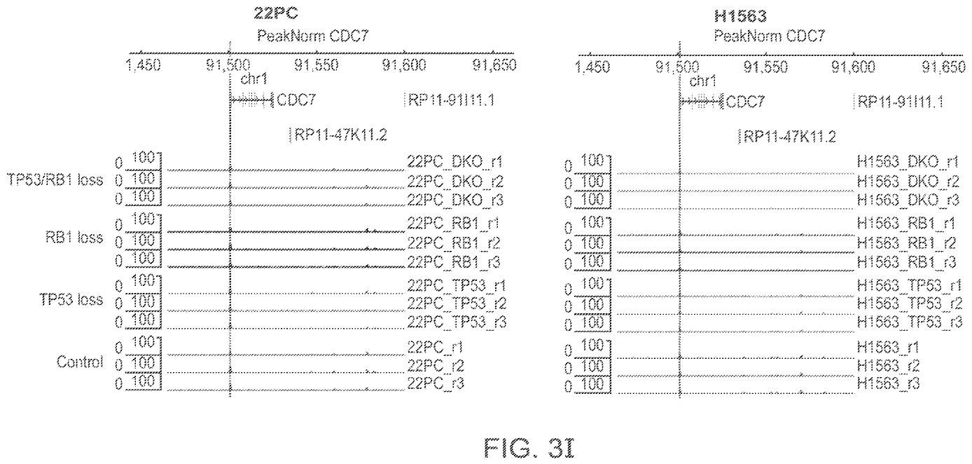

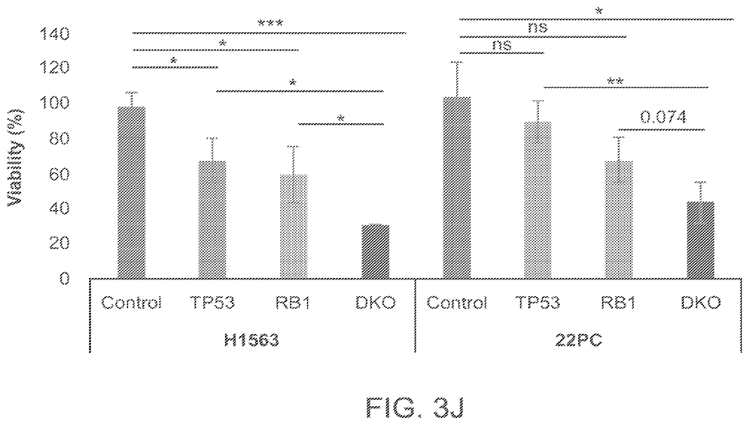

Assessment of CDC7 mRNA expression levels in NE transforming human lung tumor specimens¹ revealed upregulation of CDC7 occurring already in transforming LUADs (T-LUADs) versus control never-transformed LUAD (FIG. 3A), and further upregulation in transformed SCLC tumors (T-SCLCs). De novo SCLC tumors showed similar CDC7 mRNA levels as in transformed SCLCs (FIG. 3A). Consistently, immunohistochemical staining of CDC7 in transforming lung cancer clinical specimens and patient-derived xenografts (PDXs) confirmed increased CDC7 protein expression in transforming LUADs versus control never-transforming LUADs and further upregulation upon transformation to SCLC (FIG. 3B). In line with these results, analysis of a publicly available dataset of transcriptomic data on a cohort of human PRADs¹² revealed increased CDC7 expression on those PRADs exhibiting NE features (FIG. 3C). Consistent with these results, patient-derived xenografts (PDXs) of NEPCs exhibited higher CDC7 protein expression than those derived from PRADs (FIGS. 3D-3E). Altogether, these data further supports that NE carcinomas may be dependent on CDC7 function, and that this gene has a role in NE transformation in both lung and prostate settings.

There is evidence of molecular and treatment response dissimilarities in de novo and transformed SCLCs^{1,13}, with T-SCLCs retaining molecular features of their previous LUAD state and showing a decreased neuronal differentiation phenotype^{1,12}. Thus, the capacity of simurosertib to sensitize T-SCLCs and NEPCs to cisplatin was examined. In vitro synergy assays in Lx1042 and H660, cell lines derived from a T-SCLC and a NEPC tumor, respectively, showed high synergy between simurosertib and cisplatin (FIG. 3F). Consistent with these results, in vivo treatments of PDXs derived from a T-SCLC (Lx1042) and a NEPC (LuCAP49) highlighted the capacity of simurosertib to dramatically sensitize either model to cisplatin (FIG. 3G). In terms of tumor growth arrest, the simurosertib and cisplatin combination showed significantly superior efficacy as compared to that of cisplatin and etoposide combo, currently used in the treatment of NE-transformed lung and prostate tumors, with tumor growth inhibition (T/C) values of 35.98% versus 59.24% and 10.18% versus 50.85% for Lx1042 and LuCap49 PDX models, respectively, at control arm experimental endpoint (FIG. 3G). These results extend the potential use of simurosertib in combination with chemotherapy to T-SCLCs and NEPCs.

### Example 5: Inhibition of CDC7 Contains NE Relapse on Targeted Therapy

The above results demonstrate that CDC7 upregulation may start early in the NE transformation process and can be detected already in T-LUADs (FIGS. 3A-3B). Dysfunction of both TP53 and RB1, described to occur either by genomic alterations or protein downregulation^{1,14,15}, is thought to be a prerequisite to induce histological transdifferentiation^{14,15} Further studies were performed to assess whether loss of TP53 and RB1 is able to induce CDC7 expression in LUAD and PRAD.

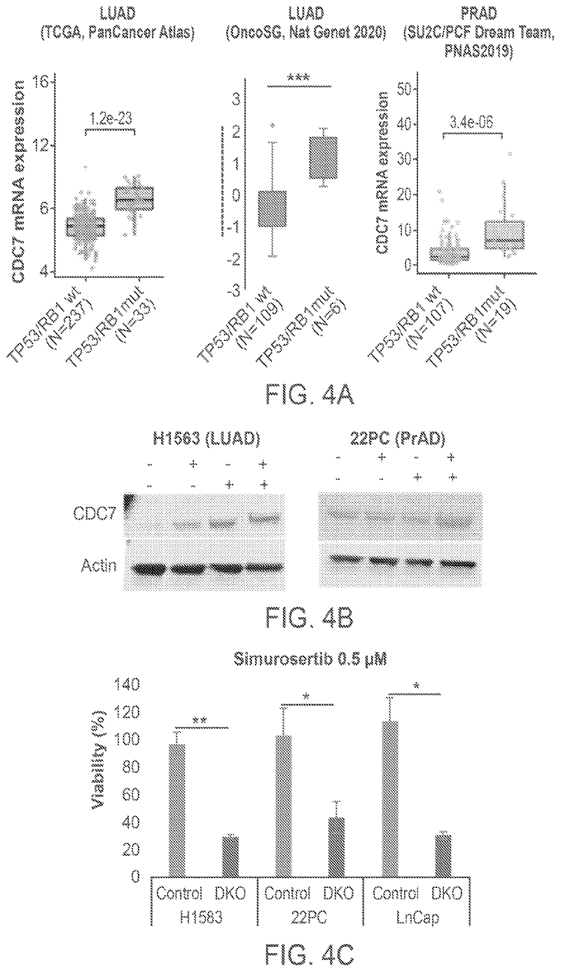

In line with this hypothesis, analysis of publicly available transcriptomic datasets of clinical specimens showed increased expression of CDC7 mRNA in LUADs and PRADs with concurrent TP53 and RBI mutations (FIG. 4A). Consistent with previous reports⁸, the data available showed increased CDC7 expression in LUADs harboring TP53 mutations but retaining RB1 function, as compared to double wild TP53/RB1-wild type tumors (FIG. 3H), suggesting that TP53 disfunction alone may be able to induce CDC7 expression in this setting (FIG. 3H). Interestingly, this was not observed in the PRAD cohort (FIG. 3H), where only samples with mutations in RB1, independently of the TP53 status, shows increased CDC7 expression to that observed in the double wild type tumors (FIG. 3H). CDC7 upregulation was observed consistently in double TP53/RB1-mutant adenocarcinomas in all cohorts under study (FIG. 2A). Then, CDC7 protein levels in TP53 and/or RB1 function loss LUAD (H1563) and PRAD (22PC) isogenic cell lines were determined by western blot (FIG. 4B). In H1563, loss of function of either gene induced CDC7 expression compared to the control condition (FIG. 4B), as opposed to the 22PC PRAD cell line, where no significant increase of CDC7 was observed after individual loss of either gene. However, consistent with the clinical data (FIG. 3H), double TP53/RB1 loss induced the highest CDC7 levels as compared to any of the other conditions under assay in both cell lines (FIG. 4B). ATAC-seq data on these cell lines did not reveal increased CDC7 gene chromatin accessibility after TP53/RB1 loss of function (FIG. 3I), suggesting an alternative mechanism of CDC7 upregulation. Importantly, increased simurosertib sensitivity was observed in the condition of double TP53/RB1 loss for these and an additional PRAD cell line (LnCap) as compared to control (FIG. 4C) or single gene loss-of-function (FIG. 3J) counterparts.

These data demonstrate that loss of TP53 and RB1 may not only result in upregulated CDC7 expression, but also in increased dependency for this gene. Together with the observation that CDC7 is upregulated already in T-LUAD (FIGS. 3A-3E), these results indicate CDC7 as a therapeutic target to interfere with NE transformation in patients with concurrent TP53 and RB1 loss, at high risk of transformation^{1,15}.

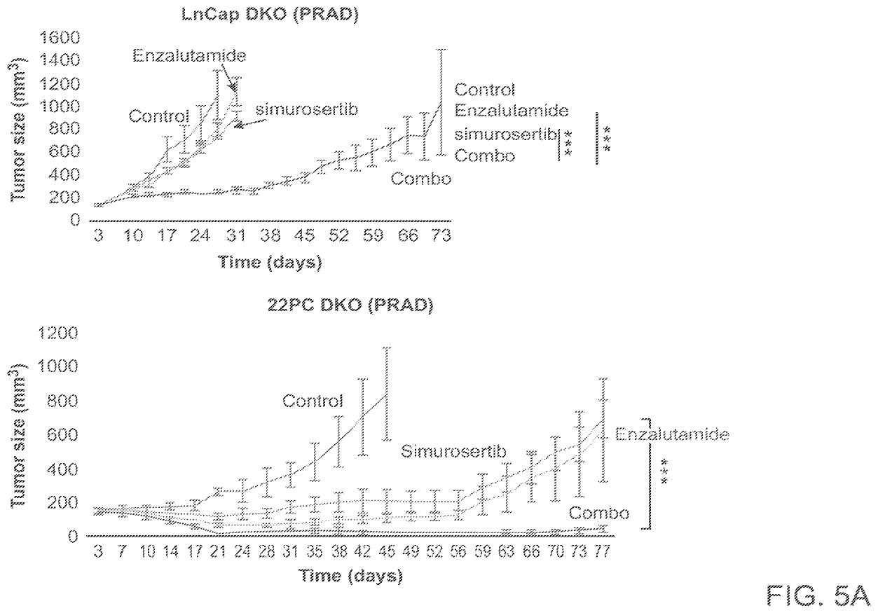

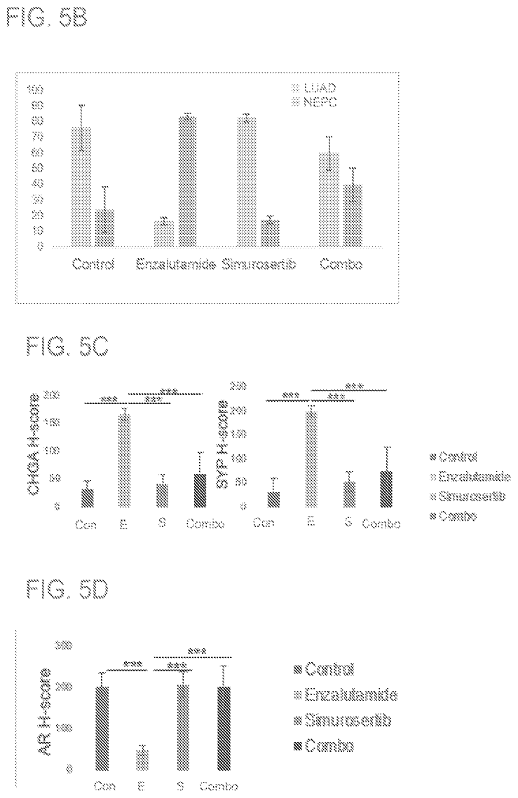

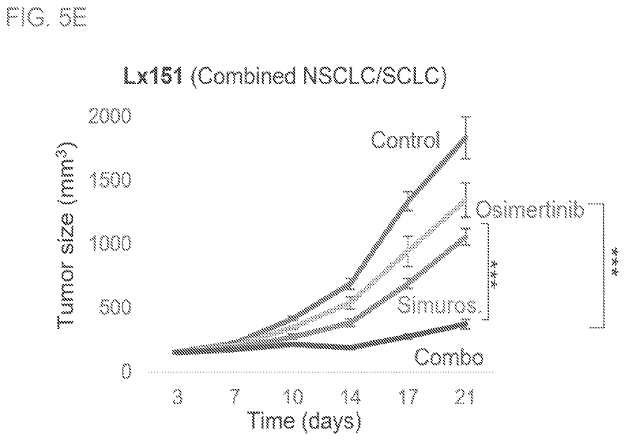

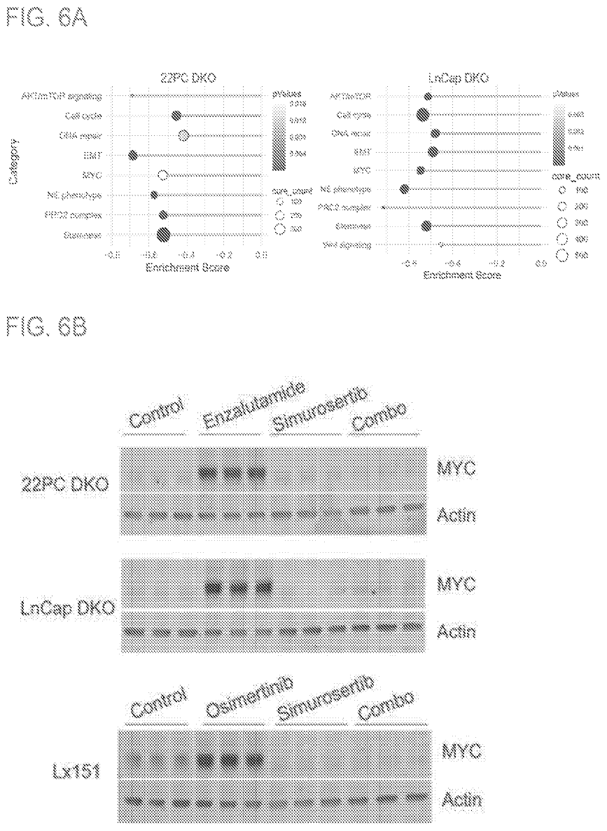

To evaluate the potential of CDC7 inhibition as a therapeutic approach to prevent NE transformation, previously described prostate NE transformation models, the PRAD cell lines LnCap and 22PC, were leveraged. Both cell lines contain a double knock out (DKO) of TP53 and RB1 that induces AR-targeted therapy resistance in vivo, together with a loss of epithelial features and increased NE marker expression¹⁶ accentuated by AR inhibition with enzalutamide. Xenografts of both models were generated in immunocompromised mice, and treated with enzalutamide, simurosertib or their combination (FIG. 5A).

The DKO LnCap xenografts showed resistance to enzalutamide (T/C value of 72.53% at control arm experimental endpoint, FIG. 5A). These tumors showed also limited sensitivity to simurosertib monotherapy (T/C values of 67.38% at control arm endpoint, FIG. 5A). However, the combination treatment showed dramatic efficacy, with a T/C value of 18.85% at control arm endpoint, and a significant delay in tumor relapse compared to either drug in monotherapy (31 days versus 73 days for enzalutamide-and combo-treated tumors, respectively, FIG. 5A). Additionally, in the 22PC model we could observe initial sensitivity to enzalutamide or simurosertib, followed by quick development of resistance to either drug in monotherapy, but the combo treatment was able to efficiently induce tumor regression and prevent development of resistance during the duration of the experiment (FIG. 5A). Notably, the combination treatment did not show increased toxicity compared to enzalutamide alone, as per mouse body weight measurement (FIG. 2H).

Histological assessment was performed on the tumors collected at endpoint for each of the treatment arms in the LnCap model to reveal that these tumors exhibited a mixed histological phenotype including PRAD and NEPC areas in each of the tumors (FIG. 5B). Quantification of the PRAD and NEPC components on these tumors showed a significant enrichment in NEPC component in the enzalutamide-treated tumors, as compared to the untreated controls. However, this phenotype was reverted in the combination-treated group, where the PRAD component was retained at a higher percentage, compared to the enzalutamide-treated tumors (FIG. 5B). Immunohistochemical quantification of the NE markers chromogranin A and synaptophysin revealed an increased NE phenotype in enzalutamide-treated tumors, again reverted by the combination treatment (FIG. 5C). In line with these results, AR protein expression was downregulated by enzalutamide treatment in these tumors. However, in the combination-treated tumors this phenotype was rescued by selinexor, where AR expression remained at similar levels as in the control untreated tumors (FIG. 5D). The histological analyses performed in FIGS. 5B-5D could not be performed for the 22PC model, due to the lack of tissue availability for the combo-treated tumors (too small to collect).

Additionally, a PDX derived from a combined EGFR-mutant NSCLC/SCLC tumor retaining both NSCLC and SCLC components (MSK_Lx151) mimicking an intermediate state of transformation was leveraged. Treatment of this PDX model with osimertinib yielded limited efficacy, with a T/C value of 73.30% at control arm experimental endpoint. In this model, simurosertib monotherapy showed increased efficacy (T/C of 38.86%, FIG. 5E), but the combination outperformed any other treatment condition under assay, with a T/C value of 20.49%.

Altogether, these results suggest that CDC7 inhibition interferes with the acquisition of a NE phenotype in models of NE transformation, and that its combination with targeted therapy is useful for preventing or delaying NE transformation in patients at high risk in lung and prostate cancers.

### Example 6: CDC7 Inhibition Attenuated NE Transformation in the Prostate and Lung by Activating the Proteasome and Inducing MYC Degradation

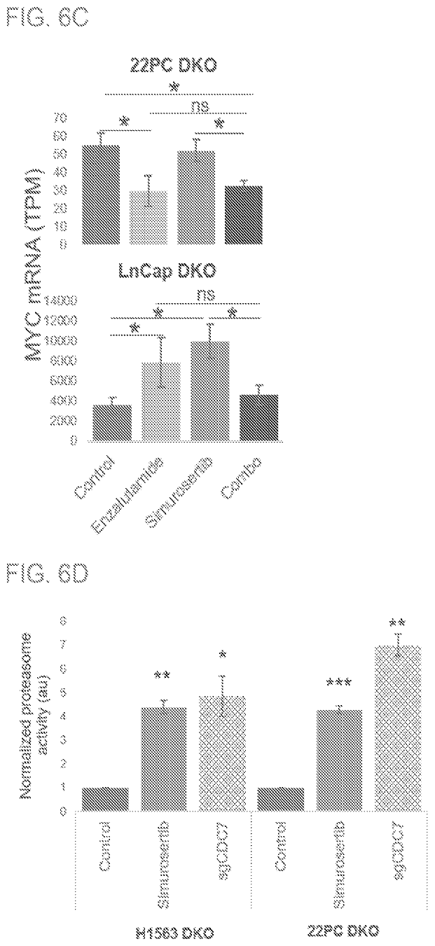

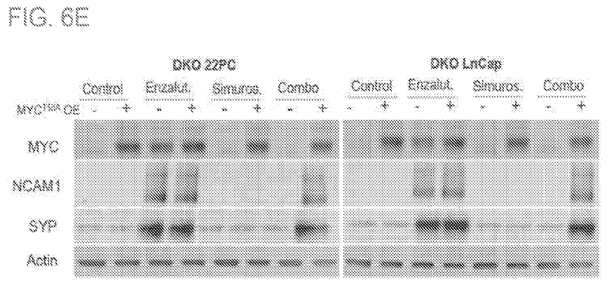

To study the mechanism by which CDC7 inhibition prevents neuroendocrine transformation, we performed RNAseq on tumors collected from experiments shown in FIG. 5A, at an intermediate time point. Pathway enrichment analyses on differentially expressed genes in the combo-versus enzalutamide-treated conditions indicated a number of pathways that were downregulated in the combo condition (FIG. 6A), including MYC, a known transcription factor involved in stemness and plasticity. We observed that MYC protein expression was upregulated in preclinical models of transformation after treatment with targeted therapy (FIG. 6B), even if the MYC mRNA levels did not show consistent downregulation (FIG. 6C). As MYC activity is tightly regulated by its degradation in the proteasome, we decided to check whether MYC protein downregulation might be linked to increased proteasome activity. Inhibition of CDC7 pharmacologically (simurosertib) or genetically (CDC7 CRISPR KO) induced proteasome activity (FIG. 6D), consistently with MYC protein downregulation in the simurosertib-treated conditions, alone or in combination with targeted therapy (FIG. 6B). To confirm that MYC downregulation induced after CDC7 inhibition was preventing neuroendocrine transformation, we treated the two prostate transformation models with enzalutamide, simurosertib and their combination and assessed expression of neuroendocrine markers. For each treatment condition, we included two isogenic cell lines, one control and one including exogenous overexpression of a MYC isoform that is resistant to proteasome degradation, MYC^{T58A} (FIG. 6E). Similarly to what we observed in vivo, enzalutamide treatment induced expression of neuroendocrine markers in these models, but these effects were reverted in the combo-treated cells. Nonetheless, combo-treated MYC^{T58A}-expressing cells retained expression of neuroendocrine markers, suggesting that MYC activity was able to revert the loss of neuroendocrine features. These results support that MYC downregulation by CDC7 inhibition is the mechanism behind the suppression of neuroendocrine transformation.

### Equivalents

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. Quintanal-Villalonga, A. et al. Multi-omic analysis of lung tumors defines pathways activated in neuroendocrine transformation. Cancer Discov candisc. 1863.2021(2021 ) doi: 10.1158/2159-8290.cd-20-1863.
2. Rudin, C. M., Brambilla, E., Faivre-Finn, C. & Sage, J. Small-cell lung cancer. Nature Reviews Disease Primers 7, (2021).
3. Zhang, H. et al. CDK7 Inhibition Potentiates Genome Instability Triggering Anti-tumor Immunity in Small Cell Lung Cancer. Cancer Cell 37, 37-54.e9 (2020).
4. Hsu, W. H. et al. Checkpoint Kinase 1 Inhibition Enhances Cisplatin Cytotoxicity and Overcomes Cisplatin Resistance in SCLC by Promoting Mitotic Cell Death. Journal of Thoracic Oncology 14, 1032-1045 (2019).
5. Misono, S. et al. Molecular signature of small cell lung cancer after treatment failure: The mcm complex as therapeutic target. Cancers 13, 1-16 (2021).
6. Sheu, Y. J. & Stillman, B. Cdc7-Dbf4 Phosphorylates MCM Proteins via a Docking Site-Mediated Mechanism to Promote S Phase Progression. Molecular Cell 24, 101-113 (2006).
7. Iwai, K. et al. Molecular mechanism and potential target indication of TAK-931, a novel CDC7-selective inhibitor. Science Advances 5, (2019).
8. Datta, A. et al. p53 gain-of-function mutations increase Cdc7-dependent replication initiation. EMBO reports 18, 2030-2050 (2017).
9. Sasi, N. K., Bhutkar, A., Lanning, N. J., MacKeigan, J. P. & Weinreich, M. DDK Promotes Tumor Chemoresistance and Survival via Multiple Pathways. Neoplasia (United States) 19, 439-450 (2017).
10. Iwai, K. et al. A CDC7 inhibitor sensitizes DNA-damaging chemotherapies by suppressing homologous recombination repair to delay DNA damage recovery. Science Advances 7, 1-15 (2021).
11. Barretina, J. et al. The Cancer Cell Line Encyclopedia enables predictive modelling of anticancer drug sensitivity. Nature 483, 603-607 (2012).
12. Abida, W. et al. Genomic correlates of clinical outcome in advanced prostate cancer. Proc Natl Acad Sci U S A 166, 11428-11436 (2019).
13. Park, C. K., Oh, I. J. & Kim, Y. C. Is transformed small cell lung cancer (SCLC) different from de novo SCLC? Transl Cancer Res 8, 346-349 (2019).
14. Niederst, M. J. et al. RB loss in resistant EGFR mutant lung adenocarcinomas that transform to small-cell lung cancer. Nat Commun 6, 199-203 (2015).
15. Offin, M. et al. Concurrent RB1 and TP53 Alterations Define a Subset of EGFR-Mutant Lung Cancers at risk for Histologic Transformation and Inferior Clinical Outcomes. Journal of Thoracic Oncology 14, 1784-1793 (2019).
16. Mu, P. et al. SOX2 promotes lineage plasticity and antiandrogen resistance in TP53- and RB1-deficient prostate cancer. Science (1979) 355, 84-88 (2017).
17. Hulton, C. H. et al. Direct genome editing of patient-derived xenografts using CRISPR-Cas9 enables rapid in vivo functional genomics. Nature Cancer 1, 359-369 (2020).
18. Park, J. W. et al. Reprogramming normal human epithelial tissues to a common, lethal neuroendocrine cancer lineage. Science 362, 91-95 (2018).
19. Quintanal-Villalonga, Á. et al. FGFR1 and FGFR4 oncogenicity depends on n-cadherin and their co-expression may predict FGFR-targeted therapy efficacy. EBioMedicine 53, 1-15 (2020).
20 Ianevski, A., Giri, A. K. & Aittokallio, T. SynergyFinder 2.0: Visual analytics of multi-drug combination synergies. Nucleic Acids Res 48, W488-W493 (2021).
21. Gardner, E. E. et al. Chemosensitive Relapse in Small Cell Lung Cancer Proceeds through an EZH2-SLFN11 Axis. Cancer Cell 31, 286-299 (2017).
22. Shrestha, C. L. et al. Cysteamine-mediated clearance of antibiotic-resistant pathogens in human cystic fibrosis macrophages. PLoS ONE 12, 1-17 (2017).
23. Patro, R., Duggal, G., Love, M. I., Irizarry, R. A. & Kingsford, C. Salmon provides fast and bias-aware quantification of transcript expression. Nature Methods 14, 417-419 (2017).
24. Zerbino, D. R. et al. Ensembl 2018. Nucleic Acids Research 46, D754-D761 (2018).
25. Pimentel, H., Bray, N. L., Puente, S., Melsted, P. & Pachter, L. Differential analysis of RNA-seq incorporating quantification uncertainty. Nat Methods 14, 687-690 (2017).
26 Chen, J. et al. Genomic landscape of lung adenocarcinoma in East Asians. Nature Genetics 52, 177-186 (2020).
27 Abeshouse, A. et al. The Molecular Taxonomy of Primary Prostate Cancer. Cell 163, 1011-1025 (2015).
28 Ritchie, M. E. et al. Limma powers differential expression analyses for RNA-sequencing and microarray studies. Nucleic Acids Research 43, e47 (2015).
29. Subramanian, A. et al. Gene set enrichment analysis: A knowledge-based approach for interpreting genome-wide expression profiles. Proc Natl Acad Sci U S A 102, 15545-15550 (2005).
30. Liberzon, A. et al. Molecular signatures database (MSigDB) 3.0. Bioinformatics 27, 1739-1740 (2011).
31. Yu, G., Wang, L. G., Han, Y. & He, Q. Y. ClusterProfiler: An R package for comparing biological themes among gene clusters. OMICS 16, 284-287 (2012).
32. Yi, L., Pimentel, H., Bray, N. L. & Pachter, L. Gene-level differential analysis at transcript-level resolution. Genome Biology 19, 1-11 (2018).
33. Langmead, B. & Salzberg, S. L. Fast gapped-read alignment with Bowtie 2. Nature Methods 9, 357-359 (2012).
34. Zhang, Y. et al. Model-based analysis of ChIP-Seq (MACS). Genome Biology 9, (2008).
35. Liao, Y., Smyth, G. K. & Shi, W. FeatureCounts: An efficient general purpose program for assigning sequence reads to genomic features. Bioinformatics 30, 923-930 (2014).
36. Love, M. I., Huber, W. & Anders, S. Moderated estimation of fold change and dispersion for RNA-seq data with DESeq2. Genome Biology 15, 1-21 (2014).
37. Lopez-Delisle, L. et al. pyGenomeTracks: reproducible plots for multivariate genomic datasets. Bioinformatics (Oxford, England) 37, 422-423 (2021).

1. A method for treating or preventing neuroendocrine tumor formation in a patient diagnosed with adenocarcinoma comprising administering to the patient (a) an effective amount of a CDC7 inhibitor or (b) an effective amount of a CDC7 inhibitor and an effective amount of at least one chemotherapeutic drug, wherein the adenocarcinoma exhibits reduced expression and/or activity of TP53 and RB1.

2. (canceled)

3. The method of claim 1, wherein the adenocarcinoma comprises (a) a genetic mutation, an indel, a copy number alteration or epigenetic downregulation in TP53 and (b) a genetic mutation, an indel, a copy number alteration or epigenetic downregulation in RB1.

4. The method of claim 1, wherein the CDC7 inhibitor and the at least one chemotherapeutic drug are administered sequentially, simultaneously, or separately.

5. The method of claim 1, wherein the at least one chemotherapeutic drug is administered orally, intranasally, parenterally, intravenously, intramuscularly, intraperitoneally, or subcutaneously, intratumorally, or topically.

6. The method of claim 1, wherein the at least one chemotherapeutic drug is an alkylating agent, a platinum agent, a taxane, a vinca agent, an aromatase inhibitor, a cytostatic alkaloid, a cytotoxic antibiotic, an antimetabolite, an endocrine/hormonal agent, or a bisphosphonate therapy agent.

7. The method of claim 1, wherein the at least one chemotherapeutic drug comprises one or more agents selected from the group consisting of cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, risedronate, pamidronate, ibandronate, alendronate, denosumab, zoledronate, tykerb, anthracyclines (e.g., daunorubicin and doxorubicin), oxaliplatin, melphalan, etoposide, mechlorethamine, bleomycin, microtubule poisons, annonaceous acetogenins, abraxane, leucovorin, nab-paclitaxel, everolimus, pegylated-hyaluronidase, pemetrexed, folinic acid, MK2206, GDC-0449, IPI-926, M402, LY293111 or combinations thereof.

8. A method for preventing neuroendocrine tumor formation in a patient diagnosed with adenocarcinoma comprising administering to the patient an effective amount of a CDC7 inhibitor and an effective amount of an androgen receptor (AR) inhibitor, optionally wherein the adenocarcinoma exhibits reduced expression and/or activity of TP53 and RB1.

9. The method of claim 8, wherein the CDC7 inhibitor and the AR inhibitor are administered sequentially, simultaneously, or separately.

10. The method of claim 8, wherein the AR inhibitor is administered orally, intranasally, parenterally, intravenously, intramuscularly, intraperitoneally, or subcutaneously, intratumorally, or topically.

11. The method of claim 8, wherein the AR inhibitor comprises one or more agents selected from the group consisting of apalutamide, bicalutamide, darolutamide, enzalutamide, flutamide, abiraterone acetate, ARN-509, and nilutamide.

12. A method for preventing neuroendocrine tumor formation in a patient diagnosed with adenocarcinoma comprising administering to the patient an effective amount of a CDC7 inhibitor and an effective amount of an epidermal growth factor receptor (EGFR) inhibitor (EGFRi), optionally wherein the adenocarcinoma exhibits reduced expression and/or activity of TP53 and RB1.

13. The method of claim 12, wherein the CDC7 inhibitor and the EGFRi are administered sequentially, simultaneously, or separately.

14. The method of claim 12, wherein the EGFRi is administered orally, intranasally, parenterally, intravenously, intramuscularly, intraperitoneally, or subcutaneously, intratumorally, or topically.

15. The method of claim 12, wherein the EGFRi comprises one or more agents selected from the group consisting of osimertinib, afatinib, erlotinib, gefitinib, icotinib, dacomitinib, rociletinib, olmutinib, cetuximab, panitumumab, nimotuzumab, and necitumumab.

16. The method of claim 8, wherein the adenocarcinoma comprises (a) a genetic mutation, an indel, a copy number alteration or epigenetic downregulation in TP53 and (b) a genetic mutation, an indel, a copy number alteration or epigenetic downregulation in RB1.

17. The method of claim 1, wherein the patient is diagnosed with TP53⁻/⁻and RB1⁻/⁻mutant adenocarcinoma, optionally wherein the TP53⁻/⁻and RB1⁻/⁻mutant adenocarcinoma is lung adenocarcinoma or prostate adenocarcinoma.

18. (canceled)

19. The method of claim 1, wherein the CDC7 inhibitor comprises one or more agents selected from the group consisting of simurosertib (TAK-931), PHA-767491, carvedilol, dequalinium chloride, ticagrelor, and clofoctol.

20. The method of claim 1, wherein the patient is non-responsive to at least one prior line of cancer therapy.

21. The method of claim 20, wherein the at least one prior line of cancer therapy is chemotherapy.

22. The method of claim 1, wherein the CDC7 inhibitor is administered orally, intranasally, parenterally, intravenously, intramuscularly, intraperitoneally, or subcutaneously, intratumorally, or topically.
